(19) United States
(12) Patent Application Publication
(10) Pub. No.: US 2025/0213470 A1
(43) Pub. Date: Jul. 3, 2025

(54) COMPOSITIONS AND METHODS

(71) Applicant: Flagship Pioneering Innovations VI, LLC, Cambridge, MA (US)

(72) Inventors: Ayse Jane MUNIZ, Cambridge, MA (US); Matthew Dean ABBINANTI, Cambridge, MA (US); Christopher Glen RAMSBORG, Cambridge, MA (US); Fabiana ZAPPALA, Cambridge, MA (US); Patrick Robert LUNDGREN, Cambridge, MA (US)

(21) Appl. No.: 18/867,277

(22) PCT Filed: Jun. 2, 2023
(86) PCT No.: PCT/US2023/067892

§ 371 Date: Nov. 19, 2024

Related U.S. Application Data
(60) Provisional application No. 63/365,791, filed on Jun. 3, 2022.

(51) Int. Cl. A61K 9/06; A61K 9/50; A61K 35/28; A61K 35/545; A61K 38/17; A61K 38/36; A61K 38/37; A61K 38/39; C12N 5/00; C12N 5/0775

(52) U.S. Cl. CPC A61K 9/06; A61K 9/5026; A61K 9/5036; A61K 35/28; A61K 35/545; A61K 38/1709; A61K 38/363; A61K 38/37; A61K 38/39; C12N 5/0012; C12N 5/0667; C12N 2533/74; C12N 2533/90

(57) ABSTRACT

The disclosure provides, inter alia, dual-layer, semi-permeable, fibrosis-resistant, hydrogel polymer enclosures, optionally containing cells, such as mammalian cells, where the enclosures are suitable for administration to a subject in need thereof and further wherein the enclosure are scalable for large volumes of delivered cells. The disclosure further provides methods of making and using the enclosures, for example, in therapeutic methods, such as treating disorders of the liver or other cells and organs treatable with, for example secretory and/or catalytic cells.

### CROSS REFERENCE TO RELATED APPLICATION

This application claims the benefit of priority to U.S. Provisional Patent Application No. 63/365,791, filed Jun. 3, 2022, the entire contents of all of which are hereby incorporated by reference.

### FIELD

The disclosure relates, in part, to dual-layer, semi-permeable, fibrosis-resistant, hydrogel polymer enclosures comprising both modified and unmodified alginates, and methods of making and using the same, that are useful for the treatment of a disease or disorder in a subject.

### BACKGROUND

Allogeneic cell therapies have been hindered for multiple reasons including foreign body response, fibrosis, and host immune response. Prior drug delivery materials fail to, during production, allow for simultaneous expansion of cells in the same chamber that serves as the delivery vehicle for implantation. Typically, cells require either genetic engineering to achieve hypoimmunity, or a physical barrier that prevents direct contact with the body. Prior anti-fibrotic alginates are limiting in that they do not prevent foreign body response arising from signals generated by the cells and fail to provide biocompatible cell enclosures. In addition, many regenerative medicine applications (e.g. liver replacement) require large cell numbers (>1 billion), but many modalities are not scalable, e.g., cannot be adapted to 3D suspension, differentiation, and expansion.

A need exists for enclosures that both support the health of enclosed cells and that are suitable for administration to a subject, e.g., by ameliorating or eliminating multiple sources of perifibrotic overgrowth: attachment of macrophages, cell-derived innate immune system response, and lymphocyte activation. Preferably such enclosures should be suitable for generation of a large scale of cells.

### SUMMARY OF THE DISCLOSURE

Accordingly, the present disclosure provides, inter alia, enclosures that both support the health and phenotypic control of enclosed cells and that are suitable for administration to a subject, e.g., by ameliorating or eliminating cell death signals, by ameliorating or eliminating multiple sources of perifibrotic overgrowth, such as attachment of macrophages, cell-derived innate immune system response, and lymphocyte activation, and further enable efficient generation of a large scale of cells. In embodiments, the described enclosures, compositions containing them, and methods of using them, eliminate or reduce the need to engineer hypoimmunity by use of a semi-permeable barrier that serves as the cell expansion and differentiation chamber as well as the conduit for in vivo delivery. Without being bound to particular theory, this significantly increases manufacturing process productivity and product control, while reducing time and cost of goods which are traditionally rate-limiting for cell therapy production.

In embodiments, the disclosure provides dual-layer, semi-permeable, fibrosis-resistant, hydrogel polymer enclosures comprising both modified and unmodified alginates, comprising 10s, 100s, or 1000s of cells (e.g., mammalian—including human—cells, such as hepatocytes, including hepatocyte-containing organoids) in the interior aqueous chamber of the enclosure, where the enclosures (and compositions containing them) are suitable for administration to a subject (such as a human) in need thereof—for example, a subject with a disorder of the liver.

In embodiments, cells useful in enclosures include, generally, secretory and/or catalytic cells, including organoids containing such secretory and/or catalytic cells (including combinations of multiple distinct types of secretory and/or catalytic cells). In embodiments, the cells are primary or stem cell-derived, such as induced pluripotent stem cell (iPSC), embryonic stem cell (ESC), or adipose tissue stem cell (ASC)-derived. In embodiments, non-limiting examples of secretory and/or catalytic cells include adipogenic cells, ASCs, adipocytes, hepatocytes, (including iPSCs-derived hepatocytes or ASC-derived hepatocytes or ESC-derived hepatocytes), islet cells (including iPSCs-derived islet cells or ASC-derived islet cells or ESC-derived islet cells), dopaminergic neurons (including iPSCs-derived dopaminergic neurons or ASC-derived dopaminergic neurons or ESC-derived dopaminergic neurons), endocrine cells (including iPSCs-derived endocrine cells or ASC-derived endocrine cells or ESC-derived endocrine cells), cells derived from xeno sources (e.g. pig), cadaver tissue, live donors (e.g. hepatocytes), and embryonic stem cells. In embodiments, the secretory and/or catalytic cells are engineered cells. In embodiments, the secretory and/or catalytic cells are unengineered cells. In embodiments, the unengineered cells are useful for cellular endogenous functions of interest, including for example: secretory and/or catalytic (e.g. absorb substrate, convert, release) functions. In embodiments, the secretory cells prevent and/or reduce the accumulation of metabolism byproducts which are not used as reserve substances. In embodiments, secretory cells are specialized cells derived from elements belonging to other tissues. In embodiments, the secretory cells have endogenous functions of interest related to the production and release of molecules which can be useful to the organism where it occurs. In embodiments, the catalytic cells are cells that modulate enzymatic activity and catalytic functions of interest. In embodiments, the catalytic cells have endogenous functions of interest related to absorbing substrates, converting substrates, and releasing a desired product or molecule.

The disclosure further provides methods of making and using the enclosures, for example, in therapeutic methods, such as treating disorders of the liver or disorders relating to disfunction of any secretory and/or catalytic cells, including those exemplified above. The disclosure also provides methods of making said enclosures and compositions containing them.

### BRIEF DESCRIPTION OF THE DRAWINGS

FIG. 1 is a cross-section schematic diagram of one embodiment of a dual layer hydrogel provided by the disclosure in which the two layers are substantially contiguous.

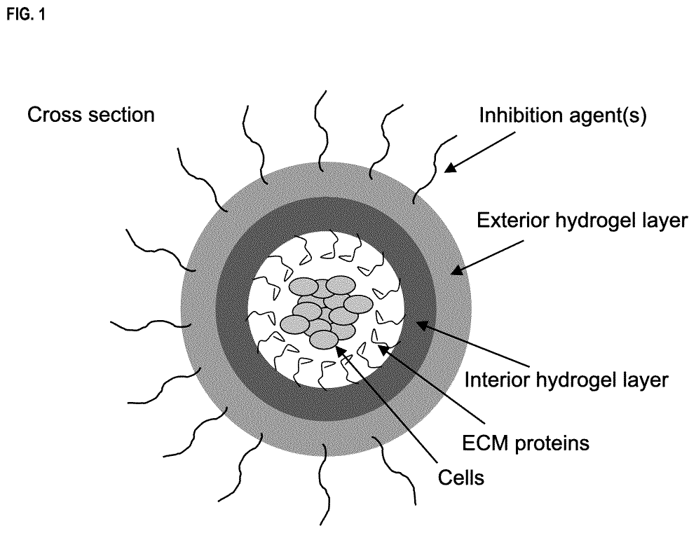

FIG. 2 is a cross-section schematic diagram of one embodiment of a dual layer hydrogel provided by the disclosure in which the two layers are separated by a fluid layer.

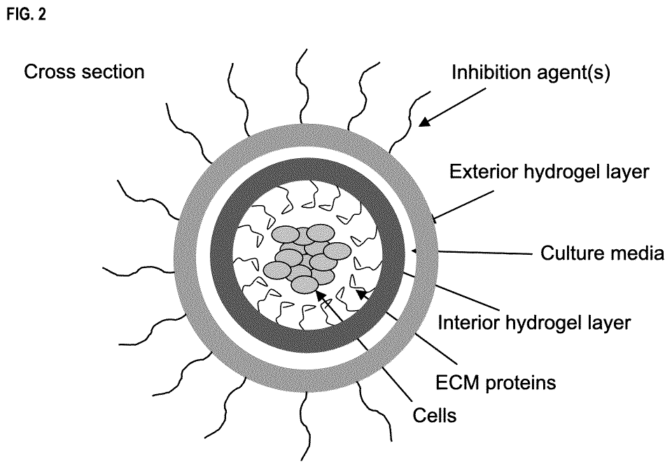

FIG. 3 is a schematic diagram of one embodiment of a method of making a dual layer hydrogel.

### DETAILED DESCRIPTION

The disclosure provides, inter alia, dual-layer, semi-permeable, fibrosis-resistant, hydrogel polymer enclosures, optionally containing cells, such as mammalian cells, where the enclosures are suitable for administration to a subject, including a human subject, in need thereof. The disclosure further provides methods of making and using the enclosures, for example, in therapeutic methods, such as treating disorders of the liver. The disclosure is based, at least in part, on applicant's discovery of fibrosis-resistant enclosures suitable for use in mammalian subject, which enclosures, without wishing to be bound by theory, can advantageously avoid fibrosis by inhibiting attachment of macrophages, cell-derived innate immune system response, and lymphocyte activation.

### Hydrogel Polymer Enclosure

In aspects, there disclosure provides a dual-layer, semi-permeable, fibrosis-resistant, hydrogel polymer enclosure comprising: an exterior hydrogel layer encompassing the enclosure; an interior hydrogel layer disposed within the exterior hydrogel layer; and an interior aqueous chamber disposed in the interior hydrogel layer, wherein the interior aqueous chamber is suitable for housing at least about 10 living mammalian cells, and wherein the hydrogel polymer enclosure is suitable for administration to a human subject. In embodiments, the exterior hydrogel layer comprises one or more rejection inhibition agents that reduce rejection upon implantation in a mammalian subject. In embodiments, the interior hydrogel layer comprises one or more agents that promote cell viability, cell differentiation, reduction and/or prevention and/or inhibition of apoptosis, cell health, and/or cell function. Non-limiting examples of cell differentiation include differentiation of a stem cell to a primary cell. In embodiments, the exterior hydrogel layer and the interior hydrogel layer are separated by a liquid layer. Non-limiting examples of a liquid layer include a culture medium (e.g. agar, alginate). In embodiments, the interior aqueous chamber has a volume allowing the at least about 10 cells to proliferate within the chamber.

In embodiments, the cells can be loaded into the hydrogel polymer enclosure as organoids, or organoid-like aggregates or spherical assemblies. In embodiments, the organoids, or organoid-like aggregates or spherical assemblies are capable of staining in suspension. In embodiments, the organoid-like aggregates or spherical assemblies are used to differentiate cells in 3D. In embodiments, the cells can be loaded into the hydrogel polymer enclosure as mini-organs. In embodiments, the mini-organ has at least two organ-specific cell types. In embodiments, the cells of the mini-organ self-organize in 3D to form structure resembling tissues in the organ. In embodiments, the mini-organ is capable of manifesting organ-specific functionality. In embodiments, the mini-organ replaces the function of an organ in the body of a subject. In embodiments, the mini-organ replaces the function of a cell in the body of a subject.

In embodiments, the cells can be loaded into the hydrogel polymer enclosure as single cells. Without being bound to a particular theory, the single cells can adhere to the hydrogel polymer enclosure's inner surfaces, the surface of any microcarriers within the hydrogel polymer enclosure, or both hydrogel polymer enclosure inner surfaces and the surface or any microcarriers within the hydrogel polymer enclosure. In embodiments, the cells can be loaded into the hydrogel polymer enclosure as organoids, or organoid-like aggregates or spherical assemblies. In embodiments, the organoids, or organoid-like aggregates or spherical assemblies are capable of staining in suspension. In embodiments, the organoid-like aggregates or spherical assemblies are used to differentiate cells in 3D. In embodiments, the cells can be loaded into the hydrogel polymer enclosure as mini-organs. In embodiments, the mini-organ has at least two organ-specific cell types. In embodiments, the cells of the mini-organ self-organize in 3D to form structure resembling tissues in the organ. In embodiments, the mini-organ is capable of manifesting organ-specific functionality. In embodiments, the mini-organ replaces the function of an organ in the body of a subject. In embodiments, the mini-organ replaces the function of a cell in the body of a subject.

In embodiments, the hydrogel polymer enclosure comprises cells in the interior aqueous chamber. In embodiments, the cells are mammalian cell, such as human cells, including cells derived from human induced pluripotent stem cells (iPSCs), adipose-derived stem cells (ASCs), or embryonic stem cells (ESCs).

In embodiments, the cells are selected from one or more of adipogenic cells, iPSCs-derived hepatocytes, adipocytes, islet cells, iPSCs-derived islet cells, iPSCs-derived dopaminergic neurons, endrocine cells, and cells derived from xeno sources (e.g. pig), cadaver tissue, live donors, adipose-derived stem cells, and embryonic stem cells.

In embodiments, the cells within the interior aqueous chamber are iPSCs, ASCs, or ESCs. In embodiments, the iPSCs, ASCs, and/or ESCs maintain an ability to divide without significant loss of or reduction of genetic stability (e.g., minimization of: copy number variation (CNV) or emergence of single-nucleotide polymorphisms (SNPs)) while maintained in culture. In embodiments, the ability of the iPSCs, ASCs, or ESCs to divide without significant loss of or reduction of genetic stability is compared to unenclosed iPSCs, ASCs, or ESCs. In embodiments, the ability of cells to divide without significant loss or reduction of genetic stability is evaluated by assays such as karyotyping, restriction endonuclease mapping, ddPCR, and/or DNA sequencing.

In embodiments, the cells within the interior aqueous chamber are iPSCs, ASCs, or ESCs and are capable of stably differentiating into mesoderm, endoderm, or ectoderm lineages.

In embodiments, the interior aqueous chamber has a volume suitable for at least about: 10, 20, 30, 40, 50, 60, 70, 80, 90, 100, 150, 200, 250, 300, 350, 400, 450, 500, 550, 600, 650, 700, 750, 800, 850, 900, 950, 1000, 1250, 1500, 2000, 2500, 3000, 3500, 4000, 4500, 5000, 6000, 7000, 8000, 9000, 10000, 11000, 12000, 13000, 14000, 15000, 16000, 17000, 18000, 19000, 20000, or more, mammalian cells (such as iPSCs, ASCs, ESCs, or cells differentiated from any of the foregoing, such as hepatocytes).

In embodiments, the interior aqueous chamber comprises at least about: 10, 20, 30, 40, 50, 60, 70, 80, 90, 100, 150, 200, 250, 300, 350, 400, 450, 500, 550, 600, 650, 700, 750, 800, 850, 900, 950, 1000, 1250, 1500, 2000, 2500, 3000, 3500, 4000, 4500, 5000, 6000, 7000, 8000, 9000, 10000, 11000, 12000, 13000, 14000, 15000, 16000, 17000, 18000, 19000, 20000, or more, mammalian cells (such as iPSCs, ASCs, ESCs, or cells differentiated from any of the foregoing, such as hepatocytes). In embodiments, the mammalian cells are genetically transformed (transiently or stably), further wherein the genetic transformation is expression of a transgene (e.g., a therapeutic protein, such as one or more of: an enzyme (including a gene editing system), a growth factor, a ligand, an antibody, or a structural protein; or a therapeutic nucleic acid, such as an siRNA or an aptamer).

In embodiments, cells maintain at least about: 80, 85, 90, 91, 92, 93, 94, 95, 96, 97, 98, 99% viability while maintaining no copy number variation over at least about: 10, 12, 15, 20, 25, 30, or more, doublings in culture (e.g., from about 1 L to 2000 L, or more) as measured via karyotyping, ddPCR, sequencing, or other assays.

In embodiments, cells maintain at least about: 80, 85, 90, 91, 92, 93, 94, 95, 96, 97, 98, 99% genetic stability as measured via karyotyping ddPCR, sequencing, or other assays during expansion over at least about: 10, 12, 15, 20, 25, 30, or more, doublings in culture (e.g., from about 1 L to 2000 L, or more).

In embodiments, at least about: 80, 85, 90, 91, 92, 93, 94, 95, 96, 97, 98, or 99%, or more, of the cells are negative for a cell death signal, such as an apoptotic or pre-apoptotic signal (such as activated Caspase 3 or Annexin V staining), e.g., over at least about: 10, 12, 15, 20, 25, 30, or more, doublings in culture (e.g., from about 1 L to 2000 L, or more) or upon administration to a subject, over at least about: 5, 10, 20, 30 days, or more, e.g., 5, 10, 15, 20 weeks. In embodiments, cell death signals include signaling activities by a cell that promote and/or result in death of the cell, including but not limited to signals such as apoptotic or pre-apoptotic signals. Non-limiting examples for identifying whether a cell is negative for a cell death signal include activated Caspase 3 or Annexin V staining.

In embodiments, the hydrogel polymer enclosures have a coefficient of variation of less than about: 15, 14, 13, 12, 11, 10, 9, 8, 7, 6, 5, 4, 3, 2, or 1%.

In embodiments, at least about: 60, 70, 80, 85, 90, 95%, or more of the hydrogel polymer enclosures contain at least about: 1, 2, 3, 4, 5, 10, 15, 20, 25, 30, 40, 50, 100, 200, 250, 500, 550, 600, 650, 700, 750, 800, 850, 900, 950, 1000, 1250, 1500, 2000, 2500, 3000, 3500, 4000, 4500, 5000, 6000, 7000, 8000, 9000, or more, mammalian cells.

A hydrogel polymer enclosure of the disclosure can be made using any method useful for preparing such enclosures, as would be understood by one of ordinary skill in the art. In embodiments, the hydrogel polymer enclosure is made by a method comprising coaxial jetting comprising an outer steam comprising a polymer solution and an interior steam comprising cells. In embodiments, the method is performed under cGMP conditions (21 CFR Parts 210, 211, 314) and/or International Council on Harmonization (ICH) quality guidelines (Q7 and others). In embodiments, the hydrogel polymer enclosure of the disclosure is made from hydrogel prepared by the methods of WO2019169245 (which is incorporated herein by reference in its entirety), e.g., including surfactants such as poloxamer 188.

In embodiments, a non-limiting example of the methods of making the hydrogel polymer enclosure of the present disclosure can be found in WO2021113751, which is incorporated herein by reference in its entirety.

In embodiments, such compositions can contain additional polymers, such as non-alginates, as well as one or more agents useful for promoting cell viability, cell function (including cell differentiation, e.g., of a stem cell to a primary cell), and/or reduction and/or prevention and/or inhibition of apoptosis, and/or preventing rejection and/or fibrosis, including by reducing (e.g., by 10, 20, 30, 40, 50, 60, 70, 80, 90, 95, 99%, or more, relative to unmodified polymers, such as alginate) or substantially eliminating one or more of macrophage attachment, cell-derived innate immune system response, or lymphocyte activation, including all three. Non-limiting examples of one or more agents include growth factors, transcription factors, proteins, and polypeptides. In embodiments, the one or more agents are useful for support cell-differentiation and/or cell viability such as extracellular matrix (ECM) proteins. In embodiments, the composition or enclosure of the present disclosure may further comprise integrated anti-inflammatory agents, including those described in WO201212982, which is incorporated herein by reference in its entirety.

In certain embodiments, one or more agents that promote cell viability and/or function useful consonant with the disclosure (e.g., on the interior hydrogel layer) is a polypeptide, or fragment thereof (e.g., a peptide of about: 5, 6, 7, 8, 9, 10, 11, 12, 13, 14, 15, 16, 17, 18, 19, 20, 25, 30, 35, 40, 45, 50, or more, contiguous residues).

In certain embodiments, the polypeptide, or fragment thereof, can include one or more of: collagen V A1, collagen V A2, collagen VI A1, collagen VI A2, collagen VI A3, fibronectin, a laminin, fibrin, fibrinogen alpha, fibrinogen beta, fibrinogen gamma, factor XIII a chain, factor XIII b chain, basement membrane-specific heparan sulfate proteoglycan core protein, elastin, including combinations of the foregoing. Exemplary sequences of these proteins, from which suitable fragments can be derived, include, but are not limited to sequences given by Uniprot accessions: P02452, P08123, P02461, P53420, P20908, P05997, P12109, P12110, P12111, P02751, P02671, P02675, P02679, P00488, P05160, P98160, P15502, Q16787, P07942, P24043, P55268, 015230, Q13751, A4D0S4, P11047, Q13753, Q16363, P08865, P25391, and Q9Y6N6, each of which is incorporated by reference in its entirety.

In embodiments, where an enclosure is made of a polymer (e.g., alginate) modified with a polypeptide, the composition is evaluated for quality, e.g., by the methods of WO2020069429, which is incorporated herein by reference in its entirety.

In embodiments, a non-limiting example of the methods of making the hydrogel polymer enclosure of the present disclosure can be found in WO2021113751, which is incorporated herein by reference in its entirety.

In embodiments, there is provided a dual-layer, semi-permeable, fibrosis-resistant, hydrogel polymer enclosure comprising: an exterior hydrogel layer encompassing the enclosure, optionally comprising one or more rejection inhibition agents that reduce rejection upon implantation in a mammalian subject; an interior hydrogel layer disposed within the exterior hydrogel layer, the interior hydrogel layer comprising alginate and comprising one or more agents that promote cell viability, cell differentiation, cell health, cell function, and/or reduction and/or prevention and/or inhibition of apoptosis, and/or preventing rejection and/or fibrosis, further optionally wherein the exterior hydrogel layer and the interior hydrogel layer are separated by a liquid layer, such as a culture medium; and an interior aqueous chamber disposed in the interior hydrogel layer, wherein the interior aqueous chamber comprises at least about 10 living induced pluripotent stem cells (iPSCs), wherein the hydrogel polymer enclosure is suitable for administration to a human subject.

In embodiments, there is provided a dual-layer, semi-permeable, fibrosis-resistant, hydrogel polymer enclosure comprising: an exterior hydrogel layer encompassing the enclosure, optionally comprising one or more rejection inhibition agents that reduce rejection upon implantation in a mammalian subject; an interior hydrogel layer disposed within the exterior hydrogel layer, the interior hydrogel layer comprising alginate and comprising one or more agents that promote cell viability, cell differentiation, cell health, cell function, and/or reduction and/or prevention and/or inhibition of apoptosis, and/or preventing rejection and/or fibrosis, further optionally wherein the exterior hydrogel layer and the interior hydrogel layer are separated by a liquid layer, such as a culture medium; and an interior aqueous chamber disposed in the interior hydrogel layer, wherein the interior aqueous chamber comprises at least about 10 living embryonic stem cells (ESCs), wherein the hydrogel polymer enclosure is suitable for administration to a human subject.

In embodiments, there is provided a dual-layer, semi-permeable, fibrosis-resistant, hydrogel polymer enclosure comprising: an exterior hydrogel layer encompassing the enclosure, optionally comprising one or more rejection inhibition agents that reduce rejection upon implantation in a mammalian subject; an interior hydrogel layer disposed within the exterior hydrogel layer, the interior hydrogel layer comprising alginate and comprising one or more agents that promote cell viability, cell differentiation, cell health, cell function, and/or reduction and/or prevention and/or inhibition of apoptosis, and/or preventing rejection and/or fibrosis, further optionally wherein the exterior hydrogel layer and the interior hydrogel layer are separated by a liquid layer, such as a culture medium; and an interior aqueous chamber disposed in the interior hydrogel layer, wherein the interior aqueous chamber comprises at least about 10 living adipose derived stem cells (ASCs), wherein the hydrogel polymer enclosure is suitable for administration to a human subject.

In embodiments, the volume allows at least about 10 cells to proliferate within the chamber. In embodiments, one or more agent is or comprises extracellular matrix (ECM) proteins. In embodiments, the cells are capable of stably differentiating into mesoderm, endoderm, or ectoderm lineages.

### Hydrogel

Any hydrogel and/or hydrogel polymer useful within enclosures of the disclosure, including the exterior hydrogel layer and/or interior hydrogel layer thereof, are contemplated herein, as would be understood by one of ordinary skill in the art. As used herein, the term “hydrogel” refers to a stable, biocompatible composition that comprises one or more polysaccharides and that swells and maintains stability in the presence of water. In embodiments, hydrogels are known to the skilled artisan and non-limiting exemplary hydrogels include alginates, acrylates (e.g. methacrylates), and/or combinations of alginates and acrylates (e.g. methacrylates), whether in admixture in one or both layers, or separate layers in the same enclosure.

In embodiments, the hydrogels are naturally derived hydrogels. Non-limiting examples of naturally derived hydrogels include but are not limited to DNA-based gels; protein-based gels (e.g. collagen, fibrin, gelatin, elastin-like peptides, fibrinogen, self-assembling peptides, elastin-like polypeptides); polysaccharide-based gels (e.g. alginate, alginate-co-gelatin, styrenated gelatin, chitosan, chondroitin sulfa, hyaluronic acid, chitin); and modified gels thereof. Non-limiting examples of modified gels include gels comprising one or more polyethylene glycol (PEG) moieties and/or one or more RGD oligopeptides. In embodiments, the hydrogels are synthetic hydrogels. Non-limiting examples of synthetic hydrogels include but are not limited to biodegradable PEG-based gels (e.g. macromers include triblock copolymers of poly(a-hydroxy esters)-b-poly(ethylene glycol)-b-poly(a-hydroxy esters) endcapped with (meth)acrylate functional groups, poly(a-hydroxy esters) (e.g. PLA, poly(8-caprolactone) (PCL)); polyfumarate-based hydrogels (e.g. macromers including poly(lactide-co-ethylene oxide-co-fumerate) and MMP-diacrylate); and phosphoester-based hydrogels (e.g. poly(6-aminohexyl propylene phosphate)-acrylate).

In embodiments, the hydrogel polymer enclosure, including the exterior hydrogel layer and/or interior hydrogel layer thereof, comprises one or more polysaccharide-based gels, one or more acrylates (e.g. methacrylate), or any combination thereof. In embodiments, the hydrogel polymer enclosure, including the exterior hydrogel layer and/or interior hydrogel layer thereof, comprises alginate. Non-limiting examples of polysaccharide-based gels include alginate, alginate-co-gelatin, styrenated gelatin, chitosan, chondroitin sulfa, hyaluronic acid, and chitin. Non limiting examples of acrylates include acrylate, methacrylate, methyl (meth)acrylate, methyl ethacrylate, ethyl (meth)acrylate, n-propyl (meth)acrylate, i-propyl (meth)acrylate, n-butyl (meth)acrylate, i-butyl (meth)acrylate, sec-butyl (meth)acrylate, tert-butyl (meth)acrylate, 2-pentyl (meth)acrylate, 3-pentyl (meth)acrylate, isopentyl (meth)acrylate, neopentyl (meth)acrylate, n-octyl (meth)acrylate, 1,1,3,3-tetramethylbutyl (meth)acrylate, ethylhexyl (meth)acrylate, n-nonyl (meth)acrylate, n-decyl (meth)acrylate, n-undecyl (meth)acrylate, tridecyl (meth)acrylate, myristyl (meth)acrylate, pentadecyl (meth)acrylate, palmityl (meth)acrylate, heptadecyl (meth)acrylate, nonadecyl (meth)acrylate, arrachinyl (meth)acrylate, behenyl (meth)acrylate, lignocerenyl (meth)acrylate, cerotinyl (meth)acrylate, melissinyl (meth)acrylate, palmitoleinyl (meth)acrylate, oleyl (meth)acrylate, linolyl (meth)acrylate, linolenyl (meth)acrylate, stearyl (meth)acrylate, lauryl (meth)acrylate, phenoxyethyl (meth)acrylate, 4-tert-butyl cyclohexylacrylate, cyclohexyl (meth)acrylate, ureido (meth)acrylate, tetrahydrofurfuryl (meth)acrylate, and mixtures thereof.

In embodiments, “alginate” encompasses both “unmodified alginate” (a polysaccharide-containing copolymer made of (1à4) linked b-D-mannuronate and alpha-L-guluronate) and “modified alginate” (comprising covalent modifications to one or more monomers of the alginate polysaccharide).

In embodiments, the exterior and interior hydrogel layers each independently comprise a polysaccharide-based gel, an acrylate, or a combination thereof. In embodiments, the interior and exterior hydrogel layers each independently comprise an alginate, a methacrylate, or a combination thereof. In embodiments, the interior and/or exterior hydrogel layers comprise an alginate or methacrylate.

In embodiments, the hydrogel polymer enclosure, including but not limited to the exterior hydrogel layer and/or the interior hydrogel layer, is further functionalized and/or modified to include additional functional groups and/or additional polymers. Non-limiting examples of additional functional groups include zwitterionic groups such as phosphorbetaine, sulfobetaine, carboxybetaine, cysteine, sulfopyridinium betaine, phosphorylcholine, or sulfobetain siloxane. Non-limiting examples of additional polymers include neutral polymers such as poly(ethylene glycol) (PEG), polysaccharides, poly(acide methacrylic) (PAAm), poly(2-hydroxyethyl methacrylate) (PHEMA), poly(N-vinyl pyrrolidone) (PVP) and poly(2-methyl-2-oxazoline) (PMOXA).

As used herein, the term “zwitterionic” refers to ionic molecule whose charge is net neutral but contains positively and negatively charged functional groups. Exemplary zwitterionic groups include, but are not limited to phosphorbetaine, sulfobetaine, carboxybetaine, cysteine, sulfopyridinium betaine, phosphorylcholine, or sulfobetain siloxane.

In embodiments, the interior and/or exterior hydrogel layers comprise an alginate and/or methacrylate, wherein the alginate and/or methacrylate comprise one or more additional functional groups and/or one or more additional polymers. In embodiments, the interior and/or exterior hydrogel layers comprise an alginate and/or methacrylate, wherein the alginate and/or methacrylate comprise one or more zwitterionic groups such as phosphorbetaine, sulfobetaine, carboxybetaine, cysteine, sulfopyridinium betaine, phosphorylcholine, or sulfobetain siloxane.

In embodiments, the hydrogel is selected from:
a) naturally derived hydrogels, optionally selected from:
   i. DNA-based gels;
   ii. Protein-based gels (e.g. collagen, fibrin, gelatin, elastin-like peptides, fibrinogen, self-assembling peptides, elastin-like polypeptides);
   iii. Polysaccharide-based gels (e.g. alginate, alginate-co-gelatin, styrenated gelatin, chitosan, Chondroitin sulfa, hyaluronic acid, chitin); and
   iv. modified gels of any one of i.-iii. (e.g. comprising one or more polyethylene glycol (PEG) moieties and/or one or more RGD oligopeptides); or
b) synthetic hydrogels, optionally selected from:
   i. Biodegradable PEG-based gels (e.g. macromers include triblock copolymers of poly(a-hydroxy esters)-b-poly(ethylene glycol)-b-poly(a-hydroxy esters) endcapped with (meth)acrylate functional groups, poly(a-hydroxy esters) (e.g. PLA, poly(8-caprolactone) (PCL));
   ii. Polyfumarate-based hydrogels (e.g. macromers including poly(lactide-co-ethylene oxide-co-fumerate) and MMP-diacrylate); and
   iii. phosphoester-based hydrogels (e.g. poly(6-aminohexyl propylene phosphate)-acrylate).

For additional examples of hydrogels useful in the disclosure, see Nicodemus and Bryant, Tissue Engineering Part B: Reviews 14 (2008), which is incorporated by reference herein in its entirety.

In embodiments, the hydrogel comprises a stiffness range of about 0.1 to about 500 kPa, e.g., about 0.1 to about 10 kPa, about 0.5 to about 15 kPa, about 1 to about 15 kPa, about 5 to about 20 kPa, about 10 to about 50 kPa, about 20 to about 100 kPa, about 150 to about 300 kPa, about 100 to about 400 kPa, about 200 to about 450 kPa or about 250 to about 500 kPa. In a further aspect, each cell containing hydrogel capsule is characterized by a stiffness of about 10 kPa, about 15 kPa, about 20 kPa, about 25 kPa, about 30 kPa, about 35 kPa, about 40 kPa, about 45 kPa, about 50 kPa, about 55 kPa, about 60 kPa, about 65 kPa, about 70 kPa, about 75 kPa, about 80 kPa, about 85 kPa, about 90 kPa, or about 95 kPa or about 100 kPa. In embodiments, the hydrogel comprises a water content of more than about 20% w/w, about 30% w/w, about 40% w/w, about 50% w/w, about 60% w/w, about 70% w/w, about 80% w/w, about 90% w/w, or about 95% w/w.

In embodiments, alginate is or comprises modified alginate. In embodiments, alginate is chemically modified, e.g., to include zwitterionic groups, including combinations of the foregoing modifications. In embodiments, an alginate composition comprises both modified and unmodified alginates in various ratios, including but not limited to a ratio of about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, or about 1:9. In embodiments, alginates comprising zwitterionic monomers can be mixed with unmodified alginates in a ratio of about 1:1, about 1:2, about 1:3, about 1:4, about 1:5, about 1:6, about 1:7, about 1:8, or about 1:9.

In embodiments, the hydrogel polymer enclosure, including but not limited to the exterior hydrogel layer and/or the interior hydrogel layer, comprises one or more rejection inhibition agents. As would be understood by one of ordinary skill in the art, rejection inhibition agents are useful to prevent undesirable protein absorption, which can result in the accumulation of undesirable proteins on the hydrogel surface. In embodiments, rejection inhibition agents can form a hydration shell to prevent undesirable protein absorption. In embodiments, inclusion of rejection inhibition agents in the hydrogel (e.g. in the exterior hydrogel layer) mitigates undesirable foreign body reactions, including but not limited to macrophage attachment and lymphocyte activation. As would be understood by one of ordinary skill in the art, foreign body reaction (FBR) is a process wherein the implant and implantation process injures the tissue around the foreign object, which triggers an inflammatory process that can develop into a fibrotic response, which envelops and isolates the implanted material. In embodiments, the exterior hydrogel layer prevents or reduces an inflammatory response during the implantation process. In a non-limiting example, the hydrogel, including but not limited to the exterior hydrogel layer and/or the interior hydrogel layer, can be modified and/or functionalized to include anti-fouling functional groups and/or anti-fouling polymers provides the hydrogel with anti-fouling properties. Examples of anti-fouling functional groups include but are not limited to zwitterionic chemical groups such as sulfobetaine, carboxybetaine, and phosphocoline. Examples of anti-fouling polymers include but are not limited to poly(ethylene glycol) (PEG), polysaccharides, poly(acide methacrylic) (PAAm), poly(2-hydroxyethyl methacrylate) (PHEMA), poly(N-vinyl pyrrolidone) (PVP) and poly(2-methyl-2-oxazoline) (PMOXA). In embodiments, the exterior hydrogel layer comprises one or more rejection inhibition agents. In embodiments, the one or more rejection inhibition agents are zwitterionic groups. In embodiments, the zwitterionic groups are selected from sulfobetaine, carboxybetaine, phosphocoline, or other anti-fouling polymers, including combinations of the foregoing.

In embodiments, the hydrogel polymer enclosure, including but not limited to the exterior hydrogel layer and/or the interior hydrogel layer, comprises one or more polyalkyelene glycol-based linkers. Non-limiting examples of polyalkyelene glycol-based linkers include polyethylene glycol (PEG), polypropylene glycol (PPG), triethylene glycol, tetraethylene glycol, pentaethylene glycol, hepatheylyene glycol, nonaethylene glycol, poly(ethylene glycol) methyl ether, poly(ethylene glycol) diacrylate (PEGDA), poly(ethylene glycol) diacrylate, (poly(ethylene glycol) diacrylate, poly(ethylene glycol) dimethacrylate (PEGDMA), and poly(ethylene glycol) dimethacrylate. In embodiments, the exterior hydrogel comprises polyethylene glycol (PEG) linkers.

In embodiments, the hydrogel polymer, including but not limited to the exterior hydrogel layer and/or the interior hydrogel layer, comprises one or more extracellular matrix (ECM) components. In a non-limiting example, inclusion of ECM components provides the hydrogel with the ability to mimic the natural extracellular matrix of various tissues. Any ECM component is contemplated by the present disclosure. Non-limiting examples of ECM components include reconstituted basement membrane (e.g., Matrigel), collagen, fibronectin, laminin, fibrin, nestin, perlecan, or signaling domains such as RGD (arginylglycylaspartic acid), including combinations thereof.

Any hydrogel polymer enclosure shape that would be useful for encapsulating cells of the disclosure is contemplated herein. In embodiments, the hydrogel polymer enclosure comprises one layer. In embodiments, the hydrogel polymer enclosure comprises a plurality of layers. In embodiments, the plurality of layers comprises one or more inner layers, one or more middle layers, and/or one or more outer layers. In embodiments, the hydrogel polymer enclosure comprises one or more inner layers, one or more middle layers, and one or more outer layers. In embodiments, the hydrogel polymer enclosure shape is capable of being modified for enhanced surface area to volume ratio, as would be understood by one of ordinary skilled the art. In embodiments, the hydrogel polymer enclosure is or comprises a coil or a cylinder. In embodiments, the hydrogel polymer enclosure comprises a sandwich of two or more layers comprising materials described herein.

In embodiments, the exterior hydrogel layer has an average thickness of about: 5-50 μm, e.g., about: 10-50 μm, about: 10-20 μm, e.g., about: 5, 10, 15, 20, 5, 30, 35, 40, 45, 50, 55, or 60 μm.

In embodiments, the interior hydrogel layer has an average thickness of about: 5-50 μm, e.g., about: 10-50 μm, about: 10-20 μm, e.g., about: 5, 10, 15, 20, 5, 30, 35, 40, 45, 50, 55, or 60 μm

In embodiments, the interior hydrogel layer and/or exterior hydrogel layer has an average pore size of about: 1-20 μm, e.g., about: 0.1, 0.25, 0.5, 0.75, 1, 2, 3, 4, 5, 6, 7, 8, 9, 10, 12, 13, 14, 15, 16, 17, 18, 19, or 20 μm, or about 1-10 μm, such as about 5 μm.

In other embodiments, the sum of the exterior hydrogel layer and interior hydrogel layer is about: 10-100 μm, e.g., about: 20-100, such as about: 20-40, e.g., about: 10, 15, 20, 5, 30, 35, 40, 45, 50, 55, 60, 65, 70, 75, 80, 85, 90, 95, or 100 μm.

In embodiments, the interior hydrogel layer and/or exterior hydrogel layer has an average pore size to allow molecules less than about: 50, 60, 70, 80, 90, 100, 150, 200, 250, 300, 350, 400, 500, 600, 700, 800, 900, or 1000 kDa to pass, but not larger molecules.

In certain embodiments, the pores are dimensioned to allow gas, fluid, and small solute (e.g., small molecules, smaller proteins) flow.

In embodiments, the hydrogel polymer enclosure is substantially spherical. In embodiments, the substantially spherical enclosure has a diameter of about: 200-1000 μm, e.g., 300-800 μm, 400-600 μm, 450-550 μm.

In embodiments, sphericity is defined as having an aspect ratio near 1 (e.g., less than about: 1.4, 1.3, 1.2, 1.19, 1.18, 1.17, 1.16, 1.15, 1.14, 1.13, 1.12, 1.11, 1.10, 1.09, 1.08, 1.07, 1.06, 1.05, 1.04, 1.03), where aspect ratio is the ratio of length to width of a sphere (as determined by, e.g., SEM).

In embodiments, the distribution of sphericity can be calculated as a coefficient of variation (CV) (e.g., less than about: 15, 14, 13, 12, 11, 10, 9, 8, 7, 6, 5, 4, 3, 2, or 1%) across multiple spheres (e.g., n≥50 spheres). In certain embodiments, substantially spherical is an aspect ratio of 1.10 with CV≤5%.

In embodiments, an alginate for use in the enclosures, compositions, and methods of the disclosure include those described in one or more of: WO2012112982, WO2012167223, WO2016019391, WO2017075631, WO2019090309, WO2018067615, WO2019169333, WO2021062263, WO2021062273, WO2022031862, WO2021119522, WO2019169245, WO2019195055, WO2020069429, WO2021113751, WO2018140834, U.S. Pat. Nos. 10,730,983, 9,867,781, 10,278,922, 10,709,667, 10,709,818, and 10,426,735, each of which is incorporated by reference in its entirety.

In embodiments, the composition or enclosure of the present disclosure may further comprise integrated anti-inflammatory agents as described in WO201212982, which is incorporated herein by reference in its entirety.

In embodiments, the hydrogel polymer enclosure comprises a modified alginate comprising one or more covalently modified monomers defined by Formula I as described in WO2012167223, which is incorporated herein by reference in its entirety.

In embodiments, the hydrogel polymer enclosure comprises a multiply modified alginate polymer having a structure according to Formula III as described in WO2016019391, which is incorporated herein by reference in its entirety.

In embodiments, the hydrogel polymer enclosure comprises a modified alginate polymer having a structure according to Formula I as described in WO2017075631, which is incorporated herein by reference in its entirety.

In embodiments, the hydrogel polymer enclosure has bound to one or more surfaces thereof a small molecule comprising a chemical moiety of Formula XII or at least one polymer comprising the chemical moiety of Formula XII as described in WO2019090309, which is incorporated herein by reference in its entirety.

In embodiments, the hydrogel polymer enclosure comprises a compound of Formula (II-C) or a pharmaceutically acceptable salt thereof as described in WO2018067615, which is incorporated herein by reference in its entirety.

In embodiments, the hydrogel polymer enclosure comprises a polymer modified with a compound of Formula (II-b), or a pharmaceutically acceptable salt thereof, as described in WO2019169333, which is incorporated herein by reference in its entirety.

In embodiments, the hydrogel polymer enclosure of the disclosure is made from hydrogel prepared by the methods of WO2019169245, e.g., including surfactants such as poloxamer 188, which is incorporated herein by reference in its entirety. In embodiments, there is provided a method of evaluating a polymer composition comprising a polymer modified with a compound of Formula (I-b) as described in WO2021062273.

In embodiments, the hydrogel polymer enclosure comprises a compound of Formula (I), or a pharmaceutically acceptable salt thereof as described in WO2022031862, which is incorporated herein by reference in its entirety.

In embodiments, the hydrogel polymer enclosure comprises a compound of Formula (I), or a pharmaceutically acceptable salt thereof, as described in WO2021119522, which is incorporated herein by reference in its entirety.

In embodiments, where an enclosure is made of a polymer (e.g., alginate) modified with a polypeptide, the composition is evaluated for quality, e.g., by the methods of WO2021062263, which is incorporated herein by reference in its entirety.

In embodiments, the hydrogel polymer enclosure comprises a particle comprising a) a first compartment, b) a second compartment; and c) a compound of Formula (I-a) as described in WO2019195055, which is incorporated herein by reference in its entirety.

In embodiments, the hydrogel polymer enclosure comprises an alginate, wherein the alginate is a high guluronic acid (G) alginate, and comprises greater than about 50%, 55%, 60%, 65%, 70%, 75%, 80%, 85%, 90%, or more guluronic acid (G). In some embodiments, the alginate is a high mannuronic acid (M) alginate, and comprises greater than about 50%, 55%, 60%, 65%, 70%, 75%, 80%, 85%, 90%, or more mannuronic acid (M). In some embodiments, the ratio of M:G is about 1. In some embodiments, the ratio of M:G is less than 1. In some embodiments, the ratio of M:G is greater than 1. In some embodiments, the alginate has an approximate molecular weight of <75 kDa, and optionally a G:M ratio of ≥1.5. In some embodiments, the alginate has an approximate molecular weight of 75 kDa to 150 kDa and optionally a G:M ratio of ≥1.5. In some embodiments, the alginate has an approximate molecular weight of 150 to 250 kDa and optionally a G:M ratio of ≥1.5. In alginate-containing particle fluid, the amount of alginate (e.g., by % weight or % volume of the fluid, relative to actual weight of the alginate) can be 0.5%, e.g., at least 0.5%, 1%, 1.5%, 2%, 2.5%, 3%, 4%, 5%, 6%, 7%, 8%, 9%, 10%, 12.5%, 15%, 20%, 25%, 30%, 35%, 40%, 45%, 50%, or more, e.g., w/w; less than 50%, e.g., less than 40%, 30%, 25%, 20%, 15%, 10%, 5%, or less. In one embodiment, the particle fluid comprises a single type of polymer. In another embodiment, the particle fluid comprises more than one type of polymer, e.g., two types of polymer or three types of polymer. In some embodiments, the particle fluid comprises an alginate. In some embodiments, the particle fluid comprises alginate and a second type of polymer (e.g., a polysaccharide, e.g., hyaluronate or chitosan). A polymer in the particle fluid may be chemically modified, for example, with a small molecule, peptide, or protein. In an embodiment, a polymer in the particle fluid (e.g., alginate) is modified with a compound comprising an amide, carboxyl, ester, amine, aryl ring, heteroaryl ring, cycloalkyl ring, a heterocyclyl ring, or a polyethylene glycol moiety. In an embodiment, a polymer in the particle fluid (e.g., alginate) is modified with a compound described in any one of WO2012112982, WO2017075630, WO2017075631, WO2017218507, or WO2018067615. In an embodiment, the compound is a small molecule compound having the following structure:

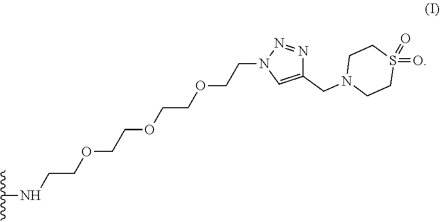 [SMILES: *NCCOCCOCCOCCn1cc(CN2CCS(=O)(=O)CC2)nn1]

In embodiments, the hydrogel polymer enclosure comprises a monomer of Formula (I) as described in WO2018140834, which is incorporated herein by reference in its entirety.

In embodiments, the hydrogel polymer enclosure comprises a biocompatible polymer comprising one or more monomer subunits A and B, wherein the polymer optionally further comprises one or more monomer subunits C; wherein each A is a zwitterionic monomer; wherein each B is a monomer with a reactive side chain, wherein the reactive side chain is Formula IV: d-R₁—Y comprising an amide bond and further comprising an ester, ether, acylhydrazine, carbamate, ketone, carbonate, sulfone, sulfoxide, thioether, azo, or aldimine; wherein each C is independently a hydrophobic monomer or a neutral hydrophilic monomer; wherein d is the point of covalent attachment of the reactive side chain to the backbone of the polymer.

In embodiments, the hydrogel polymer enclosure comprises a modified alginate polymer comprising one or more covalently modified monomers as described in Formula I of U.S. Pat. No. 10,709,818, which is incorporated by reference herein in its entirety.

In embodiments, the hydrogel polymer enclosure comprises a multiply modified alginate polymer having a structure as described in Formula III of U.S. Pat. No. 10,426,735, which is incorporated by reference herein in its entirety.

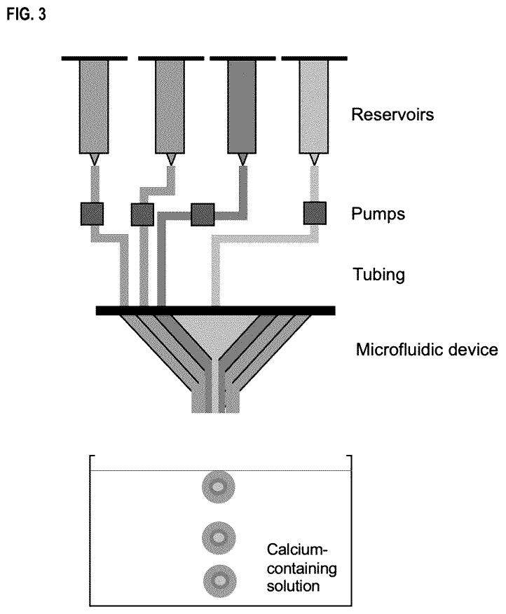

In embodiments, mixtures of these hydrogels, including the foregoing alginates, are used to feed one of three inlets of a microfluidic nozzle used in co-axial jetting, as illustrated in FIG. 3. One of the other inlet streams contains extracellular matrix molecules that form the intermediate layer which is in contact with cells. A third inlet contains cell solution. The three streams are arranged such that the cell solution is in the center, the intermediate layer surrounds the cell solution, and the alginate solution encompasses the intermediate layer and cell solution. Upon jetting of this material into a bath of calcium-containing solution, crosslinking occurs to stabilize the dual-layer hydrogel containing cells. Methods useful for making multi-layered enclosures are described in, for example, WO2018096277 (see also US2019330589), WO2018115723, WO2019101734 (see also US2020360888 and U.S. Pat. No. 11,511,254), WO2019224467 (see also US2021123013), WO2022263601, WO2022238485, each of which is incorporated by reference in its entirety.

In embodiments, there is provided a cellular microcompartment comprising successively, organized around a lumen, comprising at least one layer of human pluripotent cells; an extracellular matrix layer; and an outer hydrogel layer.

In embodiments, there is provided a chamber for culturing and imaging biological samples by optical microscopy, with said culture and imaging chamber comprising, a support and a cover, the support comprising at least one inner housing, at least one microscope slide disposed opposite an opening formed in the support or in the cover facing said inner housing; a seal disposed between the support and the cover in contact with the at least one microscope slide, the seal delimiting at least one other opening facing the microscope slide; a hydrogel layer disposed within said other opening of the seal, the hydrogel layer comprising a well array open formed at predetermined three-dimensional positions in an orthonormal frame linked to the support, each well being adapted to receive a biological sample; the support, the cover and the seal being configured to be secured to form a culture chamber and imaging sealing against a liquid.

In embodiments, there is provided a process for the manufacture of a plurality of capsules each comprising a crosslinked hydrogel outer shell surrounding a central core, wherein a hydrogel solution and an aqueous composition of interest, designed to form the central core, are concentrically coextruded to form mixed drops each comprising a layer of the hydrogel solution surrounding a drop of the composition of interest, characterized in the coextrusion step is carried out above a crosslinking aerosol so that, so that the layer of the hydrogel solution at least partially crosslinks around the drop of the composition of interest in contact with said aerosol, said capsules being spherical and having a diameter comprised between 50 and 500 μm.

### Cells

In aspects, the disclosure provides cells useful within the hydrogel polymer enclosures of the disclosure. Non-limiting examples of cells include adipogenic cells, ASCs, adipocytes, induced pluripotent stem cells (iPSCs), iPSCs-derived hepatocytes, iPSCs-derived islet cells, iPSCs-derived dopaminergic neurons, endocrine cells, cells derived from xeno sources (e.g. pig), cadaver tissue, live donors (e.g. hepatocytes), and embryonic stem cells. In embodiments, the cells are capable of stably differentiating into mesoderm, endoderm, or ectoderm lineages, including but not limited to iPSCs, ASCs, or ESCs.

In embodiments, the cells are allogenic. In embodiments, allogenic cells include cells obtained from a donor that is different from the subject to be treated. In embodiments, the secretory and/or catalytic cells are autologous.

In embodiments, the cells are substantially pure. In embodiments, substantially pure refers to a population of cells in which greater than about 80%, or greater than about 85%, greater than about 90%, or greater than about 95%, or greater than about 97%, or greater than about 98%, or greater than about 99% of the cells exhibit the same or similar characteristics (e.g., therapeutic effect, potency, differentiation capacity, mitotic activity, proliferative capacity, morphology, cell-surface markers, and combinations of the foregoing).

In embodiments, the cells are cultured and expanded. Methods of culturing are described herein, and would be understood by one of ordinary skill in the art. In embodiments, cells are cultured and expanded to the desired amount of cells. In embodiments, the secretory and/or catalytic cells are freshly prepared and/or harvested. In embodiments, the secretory and/or catalytic cells are thawed from cryopreserved stock. In embodiments, the secretory and/or catalytic cells are suitable for cryoprotection, e.g. with a cryoprotectant including, e.g. DMSO, albumin (e.g. human serum albumin) and/or saline.

In embodiments, cells are isolated from any source, as would be understood by one of ordinary skill in the art. In embodiments, the cells are isolated from adipose tissue. In embodiments, the cells are isolated from peripheral blood. In embodiments, the cells are isolated from human peripheral blood. In embodiments, the cells are mammalian cells. In embodiments, the cells are human cells. In embodiments, the cells are suitable for use in a human subject.

In embodiments, the cells are non-immunogenic. In embodiments, the cells do not trigger and/or do not substantially trigger an innate immune response in a subject. Non-limiting methods for identifying an innate immune response include measuring the level of factors indicative of an innate immune response including, but not limited to, TNFα, IFNγ, IL1β, IL6, IL10, and IL2, using any method as would be understood by one of ordinary skill in the art. In embodiments, cells of the disclosure result in no upregulation and/or substantially no upregulation of one or more factors selected from TNFα, IFNγ, IL1β, IL6, IL10, and IL2 in a subject. In embodiments, cells of the disclosure result in a reduced and/or suppressed level of one or more factors selected from TNFα, IFNγ, IL1β, IL6, IL10, and IL2 in a subject compared to a subject exhibiting an innate immune response.

In embodiments, the cells comprise or consist of adipogenic cells. Any adipogenic cells are contemplated by the present disclosure. Non-limiting examples of adipogenic cells include adipocytes, adipogenic stem cells (ASCs), and CD34⁺ cells. In embodiments, the cells are ASCs. In embodiments, the cells are derived from ASCs. Non-limiting examples of cells derived from ASCs include adipocytes.

In embodiments, the adipogenic cells are allogenic. Allogenic cells include cells obtained from a donor that is different from the subject to be treated. In embodiments, the adipogenic cells are autologous.

In embodiments, the cells comprise or consist of embryonic stem cells (ESCs). In embodiments, the cells are derived from ESCs.

In embodiments, the cells comprise or consist of iPSCs. Any iPSCs are contemplated by the present disclosure. In embodiments, the cells are derived from iPSCs. Non-limiting examples of cells derived from iPSCs include iPSCs-derived hepatocytes, iPSCs-derived islet cells, and iPSCs-derived dopaminergic neurons. Non-limiting examples of islet cells include alpha cells, beta cells, delta cells, and PP (gamma cells or F cells) cells.

In embodiments, the cells comprise or consist of endocrine cells. Any endocrine cells are contemplated by the present disclosure. In a non-limiting embodiment, endocrine cells can be derived from the pancreas, thyroid, parathyroid, pineal, pituitary, hypothalamus, ovaries, and/or testes.

In embodiments, pancreatic cells comprise or consist of alpha cells (e.g. capable of secreting glucagon hormone), delta cells (e.g. capable of secreting somatostatin hormone), and/or beta cells (e.g. capable of secreting insulin). In embodiments, pancreatic cells comprise or consist of thyroid cells, including follicular cells of the thyroid, and/or C-cells of the thyroid (e.g. capable of producing calcitonin). In embodiments, parathyroid cells comprise or consist of chief cells (e.g. capable of secreting parathyroid hormone). In embodiments, pineal cells comprise or consist of pinealocytes (e.g. capable of secreting melatonin). In embodiments, pituitary cells comprise or consist of thyrotropes (e.g. capable of secreting thyrotropin), lactotropes (e.g. capable of secreting prolactin), corticotropes (e.g. capable of secreting adrenocorticotropic hormone (ACTH)), somatotropes (e.g. capable of secreting growth hormone), and gonadotropes that (e.g. capable of secreting gonadotropins such as luteinizing hormone and follicle stimulating hormone In embodiments, hypothalamus cells comprise or consist of secretory neurons (e.g. capable of secreting anti-diuretic hormone and oxytocin). In embodiments, endocrine cells comprise or consist of endocrine cells of the ovaries and testes.

In embodiments, the cells comprise or are cells derived from xeno (xenograft) sources (e.g. pig), cadaver tissues, live donors (e.g. hepatocytes), and/or embryonic stem cells (ESCs). Any cells derived from xeno sources, cadaver tissues, live donors, and/or ESCs are contemplated by the present disclosure. Non-limiting examples of xeno sources include pig and goat.

### Methods of Treatment

In embodiments, there is provided a method or for delivering one or more cells, the method comprising contacting a biological tissue with a hydrogel polymer enclosure or composition of the present disclosure, wherein the hydrogel polymer enclosure or composition comprises one or more cells, such as mammalian cells.

In embodiments, the hydrogel polymer enclosure of the present disclosure support the health and phenotypic control of enclosed cells that are suitable for administration to a subject, e.g., by ameliorating or eliminating cell death signals, by ameliorating or eliminating multiple sources of perifibrotic overgrowth, such as attachment of macrophages, cell-derived innate immune system response, and lymphocyte activation, and further enable efficient generation of a large scale of cells

In embodiments, the tissue is a liver tissue and the one or more cells are hepatocytes (including organoid-associated hepatocytes), such as iPSC-derived, ASC-derived, or ESC-derived hepatocytes.

In embodiments, the biological tissue is in a mammalian subject. In embodiments, the mammalian subject has, or is suspected of having, a liver disease. Non-limiting examples of a liver disease include acute liver failure (ALF; including those due to or related to inherited metabolic diseases [IMDs]), chronic liver failure, and acute-on-chronic liver failure (ACLF).

In embodiments, the mammalian subject has, or is suspected of having, a disease resulting in deficient liver function or functions. Non-limiting examples of a disease resulting in deficient liver function or functions include a) a disease resulting in deficient synthesis of a protein (e.g., alpha 1 antitrypsin deficiency, Wilson's disease) or multiple proteins by the liver, b) a disease resulting in deficient metabolic function or functions of the liver (e.g., ornithine transcarbamylase deficiency, Crigler-Najjar syndrome), or c) a disease resulting in deficiencies in multiple functions of the liver (e.g., the various forms of liver failure, acute, chronic, and acute on chronic).

In embodiments, the mammalian subject has, or is suspected of having, a disease resulting in deficient liver function or functions, adipogenic cell (including ASCs or adipocytes) function or functions, islet cell function or functions, or dopaminergic neuron function or functions, selected from: a) a disease resulting in deficient synthesis of a protein or multiple proteins by: the liver (e.g., alpha 1 antitrypsin deficiency, Wilson's disease, clotting factor deficiencies, acute intermittent porphyria, and familial amyloidosis polyneuropathy), adipogenic cells (e.g. deficiency in secretion of adipokines such as in leptin-deficiency, or secretion of proteins such as lipoprotein lipase in familial chylomicronemia syndrome), islet cells (e.g. deficiency in secretion of insulin in type I diabetes or type II diabetes, and glucagon-deficiency), or dopaminergic neurons b) a disease resulting in deficient metabolic function or functions of: the liver (e.g., ornithine transcarbamylase deficiency, Crigler-Najjar syndrome, branched chain amino acid metabolism disorders (e.g. maple syrup urine disease), urea cycle disorders, familial hypercholesterolemia, glycogen storage diseases, hyperlipidemia, fatty acid transport disorders, disorders caused by mitochondrial defects (e.g. mitochondrial oxidation), peroxisome biogenesis disorders, hemochromatosis-hemosiderosis, organic acidemias, phenylketonuria, primary oxalosis, and tyrosinemia), adipogenic cells (e.g. branched chain amino acid metabolism disorders (e.g. maple syrup urine disease), urea cycle disorders, hyperlipidemia, fatty acid transport disorders, disorders caused by mitochondrial defects (e.g. mitochondrial oxidation), peroxisome biogenesis disorders, organic acidemias, lipogenesis disorders, lipid storage disorders, lipolysis disorders), islet cells, or dopaminergic neurons (e.g. diseases caused by decreased production or release of dopamine such as neurodegenerative diseases including Parkinson's disease) c) a disease resulting in deficiencies in multiple functions of: the liver (e.g., acute liver failure, acute alcohol hepatitis, acute-on-chronic liver failure, chronic liver failure, end-stage liver disease, viral hepatitis, autoimmune hepatitis, neonatal hepatitis, congenital hepatic fibrosis, cirrhosis, graft-vs-host disease, Budd-Chiari Syndrome, cystic fibrosis, biliary atresia, benign or malignant neoplasms of the liver, progressive familial intrahepatic cholestasis), adipogenic cells (e.g. congenital generalized lipodystrophy, congenital partial lipodystrophy, acquired generalized lipodystrophy, and acquired partial lipodystrophy), islet cells (e.g. type I diabetes and type II diabetes), or dopaminergic neurons (e.g. neurodegenerative diseases that compromise dopaminergic neurons such as Parkinson's Disease).

In embodiments, the hydrogel polymer enclosure is useful for treating a disease or disorder including, without limitation, acute liver failure, acute-on-chronic liver failure (ACLF), generalized lipodystrophy (GLD), partial lipodystrophy (PL), diabetes, hypothyroidism (including but not limited to hypothyroidism due to genetic causes (e.g. congenital hypothyroidism), hypertriglyceridemia, autoimmune diseases (e.g. Hashimoto's disease, atrophic thyroiditis), and/or due to surgical removal and/or radiation treatment (e.g. used for treating parathyroid tumors)), hypoparathyroidism (including but not limited to hypoparathyroidism due to genetic causes (e.g. inherited hypoparathyroidism) and/or due to surgical removal and/or radiation treatment (e.g. used for treating parathyroid tumors)), hormone deficiencies (e.g. growth hormone deficiencies), hypothalamic obesity, adrenal insufficiencies, and hypogonadism.

### Manufacturing and Cell Culturing

In embodiments, there is provided a method of culturing cells, comprising culturing, in a bioreactor, a hydrogel polymer enclosure or composition of the present disclosure, wherein the hydrogel polymer enclosure or composition comprises one or more cells.

In embodiments, the composition is sterile (free of fungal, bacterial, or archaeal cells; free of viral particles), pyrogen-free, substantially free of debris, or a combination of any or all of the foregoing.

In embodiments, there is provided a bioreactor cell culture system comprising a closed chamber containing a plurality of suspended cell microcompartments, wherein the microcompartments each comprise an outer hydrogel layer providing a cavity containing a set of self-organized cells and extracellular matrix or an extracellular matrix substitute.

As would be understood by one of ordinary skill in the art, a microcarrier can be used in methods of culturing cells of the disclosure. In embodiments, the cells are dispersed on a microcarrier (e.g. during culturing). In embodiments, the cells were previously cultured on a microcarrier (e.g. prior to enclosure in the hydrogen polymer enclosure). In embodiments, the cells were previously cultured in vitro (e.g. prior to enclosure in the hydrogen polymer enclosure). In embodiments, the cells are dispersed in the interior of an enclosure (e.g. a semi-permeable enclosure, such as an alginate enclosure). In embodiments, the semi-permeable enclosure is disassociated (i.e. the cells are released) before the cells are dispersed into the hydrogel polymer enclosure. In embodiments, the cells are cryopreserved. In embodiments, the cells are cultured using any combination of the forgoing. In a non-limiting example, the cells are cultured on a microcarrier in an enclosure, the semi-permeable enclosure is then disassociated to release the cells, which are then re-enclosed in the hydrogel polymer enclosure.

In embodiments, the cells are dispersed on a microcarrier; were previously cultured in the interior of a semi-permeable enclosure (such as an alginate enclosure), before disassociating the semi-permeable enclosure, recovering the cells, and re-enclosing the cells in the hydrogel polymer enclosure of any of the preceding Embodiments; were recovered from cryopreservation; or a combination of the foregoing.

In embodiments, the cells are encapsulated by means of solid microcarriers. In embodiments, the microcarrier is coated with a variety of bioactive materials, such as collagen, gelatin, fibrin, fibronectin, albumin, hyaluronic acid, elastin, chitosan, alginate, silk, copolymers thereof, or combinations thereof.

In embodiments, the microcarriers comprises non-resorbable polymers (e.g. polyethylene, polyethylene oxide, polyethylene terephthalate, polyester, polymethylmethacrylate, polyacrylonitrile, silicone, polyurethane (PU), polycarbonate, polyether ketone, polyether ether ketone, polyether imide, polyamide, polystyrene, polyether sulfone, polysulfone, polyvinyl acetate, polytetrafluoroethylene, polyvinylidene fluoride, copolymers thereof, or combinations thereof); resorbable polymers (e.g. polycaprolactone, poly(lactide-co-caprolactone), poly(lactide-co-glycolide), polyglycolide, polylactic acid, including derivatives thereof such as, without limitation, poly(L-lactic acid), and poly(D, L-lactic acid), polyglycolic acid, polydioxanone, poly(-hydroxybutyrate-co-3-hydroxyvalerate), trimethylene carbonate, polydiols, polyesters, polyethylene terephthalate (PET), poly(butylene terephthalate) (PBT), polyurethane, polyethylene, polyethylene oxide, polymethylmethacrylate, polyacrylonitrile, silicone, polycarbonate, polyether ketone, polyether ether ketone, polyether imide, polyamide, polystyrene, polyether sulfone, polysulfone, polyvinyl acetate, polytetrafluoroethylene, polyvinylidene fluoride, polyglycolic acid, polydioxanone, collagen, gelatin, fibrin, fibronectin, albumin, hyaluronic acid, elastin, chitosan, alginate, or combinations thereof. In embodiments, the resorbable polymers are selected from poly(lactide-co-glycolide), polyglycolide, poly(L-lactic acid), copolymers thereof, and combinations thereof); natural polymers (collagen, gelatin, fibrin, fibronectin, albumin, hyaluronic acid, elastin, chitosan, alginate, silk (e.g. silk fibroin), copolymers thereof, or combinations thereof); polyvinyl alcohol (PVA); nylon; and 1D polymer nanofibers (e.g. polyurethane, polyurethane copolymer, cellulose acetate, cellulose, acetate butyrate, cellulose derivative, styrene-acrylonitrile (SAN), polyacrylonitrile (PAN), poly(vinyl acetate) (PVAc), polyvinylpyrrolidone (PVP), polyvinyl alcohol (PVA), polyacrylic acid (PAA), hydroxypropyl cellulose (HPC), polymethylmethacrylate (PMMA), polyfurfuryl alcohol (PFA), polystyrene (PS), polystyrene copolymer, polyaniline (PANT), polyvinylchloride (PVC), polypropylene (PP)), and polyimide, or combinations thereof).

For additional examples of microcarrier cultures, see Ashok, P., Fan, Y., Rostami, M. R., & Tzanakakis, E. S. (2016). Aggregate and Microcarrier Cultures of Human Pluripotent Stem Cells in Stirred-Suspension Systems. Methods in Molecular Biology, 1502, 35-52, and “General Guidelines for Corning Microcarriers”, www.corning.com/catalog/cls/documents/protocols/protocol_CLS_AN_241_microcarriers.pdf which are incorporated by reference herein in their entireties.

In embodiments, the disclosure provides compositions useful for encapsulation, for example microencapsulation, of the compositions and/or the cells of the disclosure. In embodiments, the compositions and/or cells of the disclosure are modified by disposal and/or encapsulation in the hydrogel polymer enclosure disclosed herein.

In embodiments, the encapsulation shape is spherical. In embodiments, the encapsulation shape is non spherical, such as teardrop (i.e., spherical and elongated teardrop). In embodiments, the encapsulation shape is engineered for manufacturing. In embodiments, the encapsulation shape is formed into a scaffold, a pouch, and/or tube, or a tubular lattice.

In embodiments, the cells/organoids are removed with the interior of the composition of the present disclosure using chemical or biological means to dissociate the hydrogel without adversely affecting the cells/organoids. In embodiments chemical or biological means include using calcium chelators or alginate lyase. In embodiments, non-limiting examples of calcium chelators include EDTA, EGTA, and sodium citrate.

For additional examples of using sodium citrate to dissociate hydrogels, see Maguire, T., Novik, E., Schloss, R., & Yarmush, M. (2006). Alginate-PLL microencapsulation: effect on the differentiation of embryonic stem cells into hepatocytes. Biotechnology and Bioengineering, 93 (3), 581-591, and Belhaj, M. (2018). Enhancements in Alginate Microencapsulation Technology & Impacts on Cell Therapy Development. (Doctoral dissertation). Retrieved from scholarcommons.sc.edu/etd/4475 which are incorporated by reference herein in their entireties.

For additional examples of using sodium citrate, EDTA and EGTA to dissociate hydrogels, see Rodriguez, S., Lau, H., Corrales, N., Heng, J., Lee, S., Stiner, R., Alexander, M., & Lakey, J. R. T. (2020). Characterization of chelator-mediated recovery of pancreatic islets from barium-stabilized alginate microcapsules. Xenotransplantation, 27 (1), e12554, which is incorporated by reference herein in its entirety.

For additional examples of using EDTA to dissociate hydrogels, see Lehnert, S., & Sikorski, P. (2022). Application of Temporary, Cell-Containing Alginate Microcarriers to Facilitate the Fabrication of Spatially Defined Cell Pockets in 3D Collagen Hydrogels. Macromolecular Bioscience, 22 (1), e2100319, which is incorporated by reference herein in its entirety.

For additional examples of using alginate lyase to dissociate hydrogels, see Zhang, H., Cheng, J., & Ao, Q. (2021). Preparation of Alginate-Based Biomaterials and Their Applications in Biomedicine. Marine Drugs, 19 (5), and Saenz Del Burgo, L., Ciriza, J., Espona-Noguera, A., IIIa, X., Cabruja, E., Orive, G., Hernández, R. M., Villa, R., Pedraz, J. L., & Alvarez, M. (2018). 3D Printed porous polyamide macrocapsule combined with alginate microcapsules for safer cell-based therapies. Scientific Reports, 8 (1), 8512, which are incorporated by reference herein in their entireties.

In embodiments, a non-limiting protocol for using EDTA to dissociate hydrogels comprises using EDTA at a concentration of about 10-1000 mM, or about 50-100 mM, a buffer solution (i.e. phosphate buffered saline), and incubating the hydrogel for less than about 20 minutes, or 10 minutes at room temperature (i.e. about 37 degrees Celsius).

In embodiments, a non-limiting protocol for using sodium citrate to dissociate hydrogels comprises using sodium citrate at a concentration of about 10-1000 mM, or about 55-100 mM, a buffer solution (i.e. 10 mM HEPES on 10 mM MOPS and 27 mM NaCl), and incubating the hydrogel at about 5 minutes to 2 hours, or 5 minutes to 30 minutes shaking at room temperature (i.e. about 37 degrees Celsius).

In embodiments, a non-limiting protocol for using alginate lyase to dissociate hydrogels comprises using alginate lyase at a concentration of about 500 g/mL.

Without being bound to a particular theory, this dissociation would allow for re-encapsulation of cells/organoids if a multi-step process is required for iPSC expansion and cell differentiation.

In embodiments, cryopreservation is a process where organelles, cells, tissues, extracellular matrix, organs or any other biological constructs susceptible to damage caused by unregulated chemical kinetics are preserved by cooling to very low temperatures (typically −80° C. using solid carbon dioxide or −196° C. using liquid nitrogen). At low enough temperatures, any enzymatic or chemical activity which might cause damage to the biological material in question is effectively stopped. Cryopreservation methods seek to reach low temperatures without causing additional damage caused by the formation of ice during freezing.

In embodiments, there is provided a method comprising cooling the composition of the present disclosure to about −30, −40, −50, −60, −70, −80, −90, −100, −110, −120, −130, −140, −150, −160, −170, −180, −190 degrees Celsius.

In embodiments, a non-limiting method of cryopreservation comprises using gradual increase in dimethyl sulfoxide (DMSO) concentration prior to freezing, followed by a highly controlled supercooling process (e.g. range: about −0.25° C./min up to about −5° C./min).

In embodiments, the hydrogel polymer enclosure is suitable for cryopreservation, further comprising one or more of: DMSO, glycerol, an anti-apoptosis compound (e.g., ZVAD), an iron chelator (e.g., desferoxamine), serum albumin (such as human serum albumin), or a combination of the foregoing.

In embodiments, the hydrogel polymer enclosure is cryopreserved which enables thawing while maintaining cell viability. In embodiments, the hydrogel polymer enclosure was recovered from cryopreservation. In embodiments, the cells are suitable for cryoprotection, e.g. with a cryoprotectant including, e.g. DMSO, albumin (e.g. human serum albumin) and/or saline.

In embodiments, a non-limiting method of cryopreservation comprises using a slow cooling rate process. In embodiments, slow cooling rate for cryopreservation of mature isolated hepatocytes utilizes DMSO as the cryoprotective agent and various rates of cooling ranging from about −1° C. to about −5° C./min, or up to about −40° C. or about −80° C. before storing at −196° C. in liquid nitrogen.

For additional examples of methods of cryopreservation, see Whaley, D., Damyar, K., Witek, R. P., Mendoza, A., Alexander, M., & Lakey, J. R. (2021). Cryopreservation: An Overview of Principles and Cell-Specific Considerations. Cell Transplantation, 30, 963689721999617, Huang, H., Yarmush, M. L., & Usta, O. B. (2018). Long-term deep-supercooling of large-volume water and red cell suspensions via surface sealing with immiscible liquids. Nature Communications, 9 (1), 3201, and Jitraruch, S., Dhawan, A., Hughes, R. D., Filippi, C., Lehec, S. C., Glover, L., & Mitry, R. R. (2017). Cryopreservation of Hepatocyte Microbeads for Clinical Transplantation. Cell Transplantation, 26 (8), 1341-1354, which are incorporated by reference herein in their entireties.

In embodiments, a non-limiting example of the methods of making the hydrogel polymer enclosure of the present disclosure can be found in WO2021113751, which is incorporated herein by reference in its entirety.

In aspects, the disclosure provides a dosage form of a therapeutically effective amount of a hydrogel polymer enclosure of the disclosure, and/or compositions thereof. In embodiments, the dosage form of a therapeutically effective amount of a hydrogel polymer enclosure of the disclosure, and/or compositions thereof, is cell-containing, e.g., human cells, such as secretory or catalytic cells disclosed herein.

In embodiments, the dosage form is a container, such as a pre-filled syringe. In embodiments, the pre-filled syringe is sterile. In embodiments, the dosage form further comprises a pharmaceutically acceptable carrier, diluent, excipient, or vehicle. In embodiments, the dosage form is a syringe comprising one or more hydrogel polymer enclosures and/or compositions of the present disclosure. In some embodiments, the syringe is prefilled with a volume of the composition.

Subjects and/or Animals

In embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal, such, for example, a zebrafish. In embodiments, the subject and/or animal may comprise fluorescently-tagged cells (with e.g. GFP). In embodiments, the subject and/or animal is a transgenic animal comprising a fluorescent cell, such as, for example, an RPE cell and/or an immune cell. In embodiments, the subject and/or animal is a human. In embodiments, the human is a pediatric human. In embodiments, the human is an infant or child. In embodiments, the human is an adult human. In embodiments, the human is a geriatric human. In other embodiments, the human may be referred to as a patient.

In embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In other embodiments, the subject is a non-human animal, and therefore the disclosure pertains to veterinary use. In a specific embodiment, the non-human animal is a household pet. In another specific embodiment, the non-human animal is a livestock animal.

### Numbered Embodiments

Features of the enclosures, compositions, or methods of making and using them can include one or more of the following enumerated embodiments.

Embodiment 1: A dual-layer, semi-permeable, fibrosis-resistant, hydrogel polymer enclosure comprising:
an exterior hydrogel layer encompassing the enclosure, optionally comprising one or more rejection inhibition agents that reduce rejection upon implantation in a mammalian subject;
an interior hydrogel layer disposed within the exterior hydrogel layer, optionally comprising one or more agents that promote cell viability, cell differentiation, cell health, cell function, and/or reduction and/or prevention and/or inhibition of apoptosis, and/or preventing rejection and/or fibrosis, further optionally wherein the exterior hydrogel layer and the interior hydrogel layer are separated by a liquid layer, such as a culture medium; and
an interior aqueous chamber disposed in the interior hydrogel layer, wherein the interior aqueous chamber is suitable for housing at least about 10 living mammalian cells, optionally where the volume allows the at least about 10 cells to proliferate within the chamber,
wherein the hydrogel polymer enclosure is suitable for administration to a human subject.

Embodiment 2: The hydrogel polymer enclosure of Embodiment 1 where the interior and exterior hydrogel layers independently comprise an alginate, a methacrylate, or a combination thereof.

Embodiment 3: The hydrogel polymer enclosure of Embodiment 1, wherein the interior and/or exterior hydrogel layers comprise an alginate or methacrylate, optionally wherein the alginate or methacrylate comprises one or more zwitterionic groups such as phosphorbetaine, sulfobetaine, carboxybetaine, cysteine, sulfopyridinium betaine, phosphorylcholine, or sulfobetain siloxane.

Embodiment 4: The hydrogel polymer enclosure of any one of the preceding Embodiments, comprising cells in the interior aqueous chamber, optionally wherein the cells are mammalian cell, such as human cells, including hepatocytes, adipocytes, and islet cells, or cells derived from human induced pluripotent stem cells (iPSCs), adipose-derived stem cells (ASCs), or embryonic stem cells (ESCs).

Embodiment 5: The hydrogel polymer enclosure of Embodiment 4, wherein the cells within the interior aqueous chamber are iPSCs, ASCs, or ESCs and maintain an ability to divide without significant loss of genetic stability (e.g., minimization of: copy number variation (CNV) or emergence of single-nucleotide polymorphisms (SNPs) while maintained in culture, optionally as evaluated by assays such as karyotyping, restriction endonuclease mapping, ddPCR, and/or DNA sequencing.

Embodiment 6: The hydrogel polymer enclosure of Embodiment 4, wherein the cells within the interior aqueous chamber are iPSCs, ASCs, or ESCs and are capable of stably differentiating into mesoderm, endoderm, or ectoderm lineages.

Embodiment 7: The hydrogel polymer enclosure of any one of the preceding Embodiments, wherein the interior hydrogel layer comprises one or more agents that promote cell viability and/or function, optionally wherein the one or more agents that promote cell viability and/or function are polypeptides that have a biological function promoting genetic stability and/or facilitating differentiation, including one or more of: an extracellular matrix (ECM) component, such as one or more of: reconstituted basement membrane (e.g., Matrigel), collagen, fibronectin, laminin, fibrin, nestin, perlecan, or signaling domains such as RGD, including combinations thereof.

Embodiment 8: The hydrogel polymer enclosure of any one of the preceding Embodiments, wherein the exterior hydrogel layer comprises one or more rejection inhibition agents are zwitterionic chemical groups (e.g., modifying the hydrogel), optionally wherein the zwitterionic chemical groups prevent undesirable protein adsorption, e.g., via formation of a hydration shell.

Embodiment 9: The hydrogel polymer enclosure of Embodiment 8, wherein the zwitterionic chemical groups are selected from sulfobetaine, carboxybetaine, phosphocoline, or other anti-fouling polymers, including combinations of the foregoing.

Embodiment 10: The hydrogel polymer enclosure of Embodiment 8, wherein the exterior hydrogel comprises polyethylene glycol (PEG) linkers.

Embodiment 11: The hydrogel polymer enclosure of Embodiment 8, wherein the exterior hydrogel layer mitigates undesirable foreign body reactions including macrophage attachment and lymphocyte activation.

Embodiment 12: The hydrogel polymer enclosure of any one of the preceding Embodiments comprising an extracellular matrix (ECM) component, such as one or more of: reconstituted basement membrane (e.g., Matrigel), collagen, fibronectin, laminin, fibrin, nestin, perlecan, or signaling domains such as RGD, including combinations thereof.

Embodiment 13: The hydrogel polymer enclosure of any one of the preceding Embodiments, wherein the enclosure is substantially spherical.

Embodiment 14: The hydrogel polymer enclosure of Embodiment 13, wherein the substantially spherical enclosure has a diameter of about: 200-1000 μm, e.g., about: 300-800 μm, 400-600 μm, or 450-550 μm, such as about 500 μm.

Embodiment 15: The hydrogel polymer enclosure of any one of the preceding Embodiments, wherein the interior aqueous chamber has a volume suitable for at least about: 10, 20, 30, 40, 50, 60, 70, 80, 90, 100, 150, 200, 250, 300, 350, 400, 450, 500, 550, 600, 650, 700, 750, 800, 850, 900, 950, 1000, 1250, 1500, 2000, 2500, 3000, 3500, 4000, 4500, 5000, 6000, 7000, 8000, 9000, 10000, 11000, 12000, 13000, 14000, 15000, 16000, 17000, 18000, 19000, 20000, or more, mammalian cells (such as iPSCs, ASCs, ESCs, or cells differentiated from any of the foregoing, such as hepatocytes).

Embodiment 16: The hydrogel polymer enclosure of any one of the preceding Embodiments, wherein the interior aqueous chamber comprises at least about: 10, 20, 30, 40, 50, 60, 70, 80, 90, 100, 150, 200, 250, 300, 350, 400, 450, 500, 550, 600, 650, 700, 750, 800, 850, 900, 950, 1000, 1250, 1500, 2000, 2500, 3000, 3500, 4000, 4500, 5000, 6000, 7000, 8000, 9000, 10000, 11000, 12000, 13000, 14000, 15000, 16000, 17000, 18000, 19000, 20000, or more, mammalian cells (such as iPSCs, ASCs, ESCs, or cells differentiated from any of the foregoing, such as hepatocytes), optionally wherein the mammalian cells are genetically transformed (transiently or stably), further wherein the genetic transformation is expression of a transgene (e.g., a therapeutic protein, such as one or more of: an enzyme (including a gene editing system), a growth factor, a ligand, an antibody, or a structural protein; or a therapeutic nucleic acid, such as an siRNA or an aptamer).

Embodiment 17: The hydrogel polymer enclosure of Embodiment 16, wherein cells maintain at least about: 80, 85, 90, 91, 92, 93, 94, 95, 96, 97, 98, 99% viability while maintaining no copy number variation over at least about: 10, 12, 15, 20, 25, 30, or more, doublings in culture (e.g., from about 1 L to 2000 L, or more) as measured via karyotyping, ddPCR, sequencing, or other assays.

Embodiment 18: The hydrogen polymer enclosure of claim 16 or 17, wherein at least about 80, 85, 90, 91, 92, 93, 94, 95, 96, 97, 98, or 99%, or more, of the cells are negative for a cell death signal, such as an apoptotic or pre-apoptotic signal (e.g., activated Caspase 3 or Annexin V staining), over at least about: 10, 12, 15, 20, 25, 30, or more, doublings in culture (e.g., from about 1 L to 2000 L, or more), or upon administration to a subject, over at least about: 5, 10, 20, 30 days, or more, e.g., 5, 10, 15, or 20 weeks.

Embodiment 19: The hydrogel polymer enclosure of Embodiments 16-18, wherein cells maintain at least about: 80, 85, 90, 91, 92, 93, 94, 95, 96, 97, 98, 99% genetic stability as measured via karyotyping ddPCR, sequencing, or other assays during expansion over at least about: 10, 12, 15, 20, 25, 30, or more, doublings in culture (e.g., from about 1 L to 2000 L, or more).

Embodiment 20: The hydrogel polymer enclosure of any one of Embodiments 16-19, wherein, upon implantation in a mammalian subject, the enclosure elicits, relative to controls (unenclosed cells or enclosures that are not fibrosis-resistant), reduced levels of one or more of: macrophage attachment, cell-derived innate immune system response, or lymphocyte activation.

Embodiment 21: The hydrogel polymer enclosure of any one of Embodiments 16-20, wherein, upon implantation in a mammalian subject, the enclosure does not elicit an immune response.

Embodiment 22: The hydrogel polymer enclosure of any one of the preceding Embodiments, wherein the exterior hydrogel layer has an average thickness of about: 5-50 μm, e.g., about: 10-50 μm, such as about: 10-20 μm, e.g., about: 5, 10, 15, 20, 5, 30, 35, 40, 45, 50, 55, or 60 μm.

Embodiment 23: The hydrogel polymer enclosure of any one of the preceding Embodiments, wherein the interior hydrogel layer has an average thickness of about: 5-50 μm, e.g., about: 10-50 μm, such as about: 10-20 μm, e.g., about: 5, 10, 15, 20, 5, 30, 35, 40, 45, 50, 55, or 60 μm.

Embodiment 24: The hydrogel polymer enclosure of any one of the preceding Embodiments, wherein the interior hydrogel layer and/or exterior hydrogel layer has an average pore size of about: 1-20 μm, e.g., about: 0.1, 0.25, 0.5, 0.75, 1, 2, 3, 4, 5, 6, 7, 8, 9, 10, 12, 13, 14, 15, 16, 17, 18, 19, or 20 μm, or about 1-10 μm, such as about 5 μm.

Embodiment 25: The hydrogel polymer enclosure of any one of the preceding Embodiments, wherein the interior hydrogel layer and/or exterior hydrogel layer has an average pore size to allow molecules less than about: 50, 60, 70, 80, 90, 100, 150, 200, 250, 300, 350, 400, 500, 600, 700, 800, 900, or 1000 kDa to pass, but not larger molecules.

Embodiment 26: The hydrogel polymer enclosure of any one of the preceding Embodiments, wherein the enclosure is made by a method comprising coaxial jetting comprising an outer steam comprising a polymer solution and an interior steam comprising cells optionally wherein the method is performed under CGMP conditions (21 CFR Parts 210, 211, 314) and/or International Council on Harmonization (ICH) quality guidelines (Q7 and others).

Embodiment 27: The hydrogel polymer enclosure of any one of the preceding claims, wherein the cell differentiation comprises differentiation of a stem cell to a primary cell.

Embodiment 28: A composition comprising a plurality of the hydrogel polymer enclosures of any one of the preceding Embodiments.

Embodiment 29: The composition of Embodiment 28, wherein the hydrogel polymer enclosures have a coefficient of variation of less than about: 15, 14, 13, 12, 11, 10, 9, 8, 7, 6, 5, 4, 3, 2, or 1%.

Embodiment 30: The composition of Embodiment 28 or 29, wherein at least about: 60, 70, 80, 85, 90, 95%, or more of the hydrogel polymer enclosures contain at least about: 1, 2, 3, 4, 5, 10, 15, 20, 25, 30, 40, 50, 100, 200, 250, 500, 550, 600, 650, 700, 750, 800, 850, 900, 950, 1000, 1250, 1500, 2000, 2500, 3000, 3500, 4000, 4500, 5000, 6000, 7000, 8000, 9000, or more, mammalian cells, cells, or more.

Embodiment 31: The composition of any one of Embodiments 28-30, wherein the composition is sterile (free of fungal, bacterial, or archaeal cells; free of viral particles), pyrogen-free, substantially free of debris, or a combination of any or all of the foregoing.

Embodiment 32: A method for delivering one or more cells, the method comprising contacting a biological tissue with a hydrogel polymer enclosure or composition of any one of the preceding Embodiments, wherein the hydrogel polymer enclosure or composition comprises one or more cells.

Embodiment 33: The method of Embodiment 32, wherein the biological tissue is in a mammalian subject.

Embodiment 34: A method for treating a disease or disorder, the method comprising providing a therapeutically effective amount of a hydrogel polymer enclosure or composition of any one of the preceding Embodiments to a tissue in a subject in need thereof, wherein the hydrogel polymer enclosure or composition comprises one or more cells.

Embodiment 35: The method of Embodiment 34, wherein the tissue is a liver tissue and the one or more cells are hepatocytes (including organoid-associated hepatocytes), such as iPSC-derived, ASC-derived, or ESC-derived hepatocytes.

Embodiment 36: The method of Embodiment 34 or 35, wherein the mammalian subject has, or is suspected of having, a liver disease, such as acute liver failure (ALF; including those due to or related to inherited metabolic diseases (IMDs)), chronic liver failure, and acute-on-chronic liver failure (ACLF).

Embodiment 37: The method of Embodiment 34 or Embodiment 35, wherein the mammalian subject has, or is suspected of having, a disease resulting in deficient liver function or functions, adipogenic cell (including ASCs or adipocytes) function or functions, islet cell function or functions, or dopaminergic neuron function or functions, selected from:
a) a disease resulting in deficient synthesis of a protein or multiple proteins by:
the liver (e.g., alpha 1 antitrypsin deficiency, Wilson's disease, clotting factor deficiencies, acute intermittent porphyria, and familial amyloidosis polyneuropathy),
adipogenic cells (e.g. deficiency in secretion of adipokines such as in leptin-deficiency, or secretion of proteins such as lipoprotein lipase in familial chylomicronemia syndrome),
islet cells (e.g. deficiency in secretion of insulin in type I diabetes or type II diabetes, and glucagon-deficiency),
or dopaminergic neurons
b) a disease resulting in deficient metabolic function or functions of:
the liver (e.g., ornithine transcarbamylase deficiency, Crigler-Najjar syndrome, branched chain amino acid metabolism disorders (e.g. maple syrup urine disease), urea cycle disorders, familial hypercholesterolemia, glycogen storage diseases, hyperlipidemia, fatty acid transport disorders, disorders caused by mitochondrial defects (e.g. mitochondrial oxidation), peroxisome biogenesis disorders, hemochromatosis-hemosiderosis, organic acidemias, phenylketonuria, primary oxalosis, and tyrosinemia),
adipogenic cells (e.g. branched chain amino acid metabolism disorders (e.g. maple syrup urine disease), urea cycle disorders, hyperlipidemia, fatty acid transport disorders, disorders caused by mitochondrial defects (e.g. mitochondrial oxidation), peroxisome biogenesis disorders, organic acidemias, lipogenesis disorders, lipid storage disorders, lipolysis disorders),
islet cells, or
dopaminergic neurons (e.g. diseases caused by decreased production or release of dopamine such as neurodegenerative diseases including Parkinson's disease)
c) a disease resulting in deficiencies in multiple functions of:
the liver (e.g., acute liver failure, acute alcohol hepatitis, acute-on-chronic liver failure, chronic liver failure, end-stage liver disease, viral hepatitis, autoimmune hepatitis, neonatal hepatitis, congenital hepatic fibrosis, cirrhosis, graft-vs-host disease, Budd-Chiari Syndrome, cystic fibrosis, biliary atresia, benign or malignant neoplasms of the liver, progressive familial intrahepatic cholestasis),
adipogenic cells (e.g. congenital generalized lipodystrophy, congenital partial lipodystrophy, acquired generalized lipodystrophy, and acquired partial lipodystrophy),
islet cells (e.g. type I diabetes and type II diabetes), or
dopaminergic neurons (e.g. neurodegenerative diseases that compromise dopaminergic neurons such as Parkinson's Disease).

Embodiment 38: A method of culturing cells, comprising culturing, in a bioreactor, a hydrogel polymer enclosure or composition of any one of the preceding Embodiments, wherein the hydrogel polymer enclosure or composition comprises one or more cells.

Embodiment 39: The composition of Embodiment 30 or 31 or the method of any one of Embodiments 32-38, wherein the cells: are dispersed on a microcarrier; were previously cultured in the interior of a semi-permeable enclosure (such as an alginate enclosure), before disassociating the semi-permeable enclosure, recovering the cells, and re-enclosing the cells in the hydrogel polymer enclosure of any of the preceding Embodiments; were recovered from cryopreservation; or a combination of the foregoing.

Embodiment 40: The composition of Embodiment 30 or 31 or the method of any one of Embodiments 32-39, wherein the hydrogel polymer enclosure was recovered from cryopreservation.

Embodiment 41: The hydrogel polymer enclosure of any one of Embodiments 1-27, compositions of any one of Embodiments 28-31, 39, or 40, wherein the composition is suitable for cryopreservation, wherein the composition optionally further comprises one or more of: DMSO, glycerol, an anti-apoptosis compound (e.g., ZVAD), an iron chelator (e.g., desferoxamine), serum albumin (such as human serum albumin), or a combination of the foregoing.

Embodiment 42: A method comprising cooling the enclosure or composition of Embodiment 41 to about: −20, −30, −40, −50, −60, −70, −80, −90, −100, −110, −120, −130, −140, −150, −160, −170, −180, −190 degrees Celsius.

Embodiment 43: A dual-layer, semi-permeable, fibrosis-resistant, hydrogel polymer enclosure comprising:
an exterior hydrogel layer encompassing the enclosure, optionally comprising one or more rejection inhibition agents that reduce rejection upon implantation in a mammalian subject;
an interior hydrogel layer disposed within the exterior hydrogel layer, the interior hydrogel layer comprising alginate and comprising one or more agents that promote cell viability, cell differentiation, cell health, cell function, and/or reduction and/or prevention and/or inhibition of apoptosis, and/or preventing rejection and/or fibrosis, further optionally wherein the exterior hydrogel layer and the interior hydrogel layer are separated by a liquid layer, such as a culture medium; and
an interior aqueous chamber disposed in the interior hydrogel layer, wherein the interior aqueous chamber comprises at least about 10 living induced pluripotent stem cells (iPSCs),
wherein the hydrogel polymer enclosure is suitable for administration to a human subject.

Embodiment 44: A dual-layer, semi-permeable, fibrosis-resistant, hydrogel polymer enclosure comprising:
an exterior hydrogel layer encompassing the enclosure, optionally comprising one or more rejection inhibition agents that reduce rejection upon implantation in a mammalian subject;
an interior hydrogel layer disposed within the exterior hydrogel layer, the interior hydrogel layer comprising alginate and comprising one or more agents that promote cell viability, cell differentiation, cell health, cell function, and/or reduction and/or prevention and/or inhibition of apoptosis, and/or preventing rejection and/or fibrosis, further optionally wherein the exterior hydrogel layer and the interior hydrogel layer are separated by a liquid layer, such as a culture medium; and
an interior aqueous chamber disposed in the interior hydrogel layer, wherein the interior aqueous chamber comprises at least about 10 living embryonic stem cells (ESCs),
wherein the hydrogel polymer enclosure is suitable for administration to a human subject.

Embodiment 45: A dual-layer, semi-permeable, fibrosis-resistant, hydrogel polymer enclosure comprising:
an exterior hydrogel layer encompassing the enclosure, optionally comprising one or more rejection inhibition agents that reduce rejection upon implantation in a mammalian subject;
an interior hydrogel layer disposed within the exterior hydrogel layer, the interior hydrogel layer comprising alginate and comprising one or more agents that promote cell viability, cell differentiation, cell health, cell function, and/or reduction and/or prevention and/or inhibition of apoptosis, and/or preventing rejection and/or fibrosis, further optionally wherein the exterior hydrogel layer and the interior hydrogel layer are separated by a liquid layer, such as a culture medium; and
an interior aqueous chamber disposed in the interior hydrogel layer, wherein the interior aqueous chamber comprises at least about 10 living adipose derived stem cells (ASCs),
wherein the hydrogel polymer enclosure is suitable for administration to a human subject.

Embodiment 46: The hydrogel polymer enclosure of any one of Embodiments 43-45, wherein the volume allows at least about 10 cells to proliferate within the chamber.

Embodiment 47: The hydrogel polymer enclosure of any one of Embodiments 43-45, wherein one or more agent is or comprises extracellular matrix (ECM) proteins.

Embodiment 48: The hydrogel polymer enclosure of any one of Embodiments 43-45, wherein the cells are capable of stably differentiating into mesoderm, endoderm, or ectoderm lineages.

Embodiment 49: A dosage form of a therapeutically effective amount of the hydrogel polymer enclosure of any one of Embodiments 1-27, or a composition of any one of Embodiments 28-31, 39, or 40.

Embodiment 50: The dosage form of Embodiment 49, wherein the dosage form is a container, optionally a syringe.

Embodiment 51: The dosage form of Embodiment 49 or 50, wherein the hydrogel polymer enclosure or composition further comprises a pharmaceutically acceptable carrier, diluent, excipient, or vehicle.

Embodiment 52: The hydrogel polymer enclosure of any one of claim 1-27, 41, or 43-48, the composition of any one of claim 28-31, 39, or 40, or method of any one of claims 32-38, 42, wherein the enclosure was made by microfluidic co-axial jetting.

It should be understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

For all patents, applications, or other reference cited herein, such as non-patent literature and reference sequence information, it should be understood that they are incorporated by reference in their entirety for all purposes as well as for the proposition that is recited. Where any conflict exists between a document incorporated by reference and the present application, this application will control. All information associated with reference gene sequences disclosed in this application, such as GenelDs or accession numbers (typically referencing NCBI or Uniprot accession numbers), including, for example, genomic loci, genomic sequences, functional annotations, allelic variants, and reference mRNA (including, e.g., exon boundaries or response elements) and protein sequences (such as conserved domain structures), as well as chemical references (e.g., PubChem compound, PubChem substance, or PubChem Bioassay entries, including the annotations therein, such as structures and assays, et cetera), are hereby incorporated by reference in their entirety.

Headings used in this application are for convenience only and do not affect the interpretation of this application.

Preferred features of each of the aspects provided by the disclosure are applicable to all of the other aspects of the disclosure mutatis mutandis and, without limitation, are exemplified by the dependent claims (or embodiments) and also encompass combinations and permutations of individual features (e.g., elements, including numerical ranges and exemplary embodiments) of particular embodiments and aspects of the disclosure, including the working examples. For example, particular experimental parameters exemplified in the working examples can be adapted for use in the claimed disclosure piecemeal without departing from the disclosure. For example, for materials that are disclosed, while specific reference of each of the various individual and collective combinations and permutations of these compounds may not be explicitly disclosed, each is specifically contemplated and described herein. Thus, if a class of elements A, B, and C are disclosed as well as a class of elements D, E, and F and an example of a combination of elements A-D is disclosed, then, even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-groups of A-E, B-F, and C-E are specifically contemplated and should be considered disclosed from disclosure of A, B, and C; D, E, and F; and the example combination A-D. This concept applies to all aspects of this application, including elements of a composition of matter and steps of method of making or using the compositions.

The forgoing aspects of the disclosure, as recognized by the person having ordinary skill in the art following the teachings of the specification, can be claimed in any combination or permutation to the extent that they are novel and non-obvious over the prior art-thus, to the extent an element is described in one or more references known to the person having ordinary skill in the art, they may be excluded from the claimed disclosure by, inter alia, a negative proviso or disclaimer of the feature or combination of features.

### EXAMPLES

### Example 1: Manufacturing of Dual-Layer Hydrogel Encapsulation of Cells

This example demonstrates, inter alia, the ability to manufacture the dual-layer hydrogel polymer enclosure containing iPSCs or ESCs for the purpose of expansion and/or differentiation and delivery as a treatment for disease.

Therapeutic design: The iPSCs or ESCs are cultured and prepared as individual cells and the solution containing these cells is pumped through a microfluidic nozzle concentrically surrounded by a second stream. The second stream contains the hydrogel containing proteins to support cell-differentiation and/or cell viability such as extracellular matrix (ECM) proteins. The design optionally includes a third concentric stream of cell culture media. The fourth (or third in the absence of the third stream described above) concentric stream flowing through the microfluidic nozzle and surrounding the previously described streams contains a modified alginate solution in which the alginate is modified to mitigate immune response when administered. These streams are pumped through the microfluidic nozzle at a rate so that a stream of dual-layered hydrogel spheres containing 10s-100s of cells are extruded into cell culture media containing calcium to polymerize the external alginate spheres.

Encapsulated cells are cultured for 10-40 days in culture media to support differentiation into hepatocytes. Without being bound to a particular theory, encapsulated cells may be disassociated and re-encapsulated to support high proliferation numbers or to allow changes to differentiation signals of the internal layer.

### Example 2: In Vitro Testing of Dual-Layer Hydrogel Encapsulation of iPSC-Derived Hepatocytes

This example demonstrates, inter alia, the in vitro activity of encapsulated cells after differentiation into iPSC-derived hepatocytes.

Therapeutic design: The encapsulated cells generated in Example 1 are evaluated for viability, ammonia metabolism, and CYP450 activity

### Materials/Methods:

Trypan blue exclusion (TPE; ThermoFisher, 15250061) is utilized to determine cell viability as described by Gramignoli, et al., 2012 (Cell Transplant. 21 (6): 1245-1260; 2012). Most encapsulated cells (>90%) examined by microscopy are unstained by trypan blue, indicating cell viability of >90%.

Ammonia metabolism is evaluated by a commercial quantitative, colorimetric ammonia metabolism assay (Millipore Sigma, AA0100). A sample containing 100 uL of encapsulated cell solution, a negative control containing 100 uL water, and 100 uL of L-Glutamate dehydrogenase solution is run according to the assay protocol. The encapsulated cell solution shows significant ammonia metabolism.

CYP450 activity is evaluated using a commercial P450-Glo assay (Promega, V9001) as described by Gramignoli, et al., 2014 (Cell Transplant. 23 (9): 1143-1151; 2014). Luciferase-IPA solution is added to wells containing encapsulated cells, a negative control (cell culture media only) and a positive control (P450 enzyme solution) and incubated for 30-90 minutes. The P450-Glo reagent is added to all wells and equilibrated for 20 minutes. The luminescence is then measured as a direct indicator of CYP450 activity. The encapsulated iPSC-derived hepatocytes demonstrate CYP450 activity above the level of the negative control.

### Example 3: In Vivo Testing of Dual-Layer Hydrogel Encapsulated iPSC-Derived Hepatocytes

This example demonstrates, inter alia, the in vivo activity of encapsulated cells after differentiation into iPSC-derived hepatocytes.

Therapeutic design: The encapsulated cells generated in Example 1 are evaluated for their in vivo activity in the acetaminophen-induced liver injury mouse model.

### Materials/Methods:

The mouse model is generated as described by Mossanen and Tacke (Laboratory Animals 49 (1): 30-36; 2015) and Viswanathan et al. (FASEB J. 35 (4): e21471; 2021). Ten- to twelve-weeks old male and female C57BL/6 mice are fasted for between 12 and 16 hours with free access to water. At the end of fasting, the mice receive an acetaminophen (N-acetyl-p-aminophenol [APAP]) dose of 300 mg/kg body weight intraperitoneally.

Two to three hours after the APAP dosing, a solution containing 50 million total encapsulated cells as generated in Example 1 are administered intraperitoneally to each mouse. Sham mice receive the APAP dose but empty dual-layer hydrogel spheres. At 24 and 48 hours, the survival rates of treated and sham mice are compared. Mice that received encapsulated cells have improved survival rates at 24 and 48 hours compared to sham mice.

### Example 4: Manufacturing of Dual-Layer Hydrogel Encapsulation of Human ASCs

This example demonstrates, inter alia, the ability to manufacture the dual-layer hydrogel polymer enclosure containing ASCs for the purpose of expansion and characterization.

In this example, ASCs from a healthy donor are obtained and prepared as individual cells. The solution containing these cells is pumped through a microfluidic nozzle concentrically surrounded by a second stream as described in Example 1. The second stream contains the hydrogel containing proteins to support cell-differentiation and/or cell viability such as extracellular matrix (ECM) proteins. The design optionally includes a third concentric stream of cell culture media. The fourth (or third in the absence of the third stream described above) concentric stream flowing through the microfluidic nozzle and surrounding the previously described streams contains a modified alginate solution in which the alginate is modified to mitigate immune response when administered. These streams are pumped through the microfluidic nozzle at a rate so that a stream of dual-layered hydrogel spheres containing 10s-100s of cells are extruded into cell culture media containing calcium to polymerize the external alginate spheres. Encapsulated cells are then expanded in culture media. To support high proliferation numbers, encapsulated cells optionally may be disassociated and re-encapsulated.

The isolated and expanded cells are decapsulated and characterized for ASCs' surface markers using flow cytometric analysis. Specifically, cells are stained with directly conjugated antibodies against CD29, CD73, CD90, CD105, CD31, CD45, and CD34. It is expected that the isolated cells will show high expression of CD29, CD73, CD90, and CD105, low expression of CD31 and CD45, and variable expression of CD34.

Overall, this example demonstrates, inter alia, the ability to manufacture the dual-layer hydrogel polymer enclosure containing ASCs for the purpose of expansion and characterization.

### Example 5: Manufacturing of Dual-Layer Hydrogel Encapsulation of Adipocytes

This example demonstrates, inter alia, the ability to manufacture the dual-layer hydrogel polymer enclosure containing ASCs for the purpose of expansion, differentiation to adipocytes, and characterization.

In this example, ASCs from a healthy donor are obtained. The ASCs are prepared, encapsulated, and expanded as described in Example 4. Differentiation to adipocytes then begins. Adipocyte Induction Media is prepared in DMEM/F12 (Thermo Fisher, 10565-018) containing 3% FBS (Gemini, 100-106), 1× Penicillin-Streptomycin (Thermo Fisher, 15140-122), 33 μM Biotin (Fisher Scientific, BP232-1), 17 μM Pantothenate (Fisher Scientific, AAA1660922), 1 μM Insulin (sigma, 19278), 187.5 μM IBMX (Fisher Scientific, AAJ64598MC), 200 μM Indomethacin (Fisher Scientific, AAA1991006), and 1 μM Dexamethasone (Fisher Scientific, D16911G) then sterile filtered through a 0.22 μM PES filter bottle. ASCs are then exposed to freshly prepared Adipocyte Induction Media and cultured for 3 days. After 3 days, sufficient Adipocyte Maintenance Media is prepared in DMEM/F12 (Thermo Fisher, 10565-018) containing 3% FBS (Gemini, 100-106), 1× Penicillin-Streptomycin (Thermo Fisher, 15140-122), 33 μM Biotin (Fisher Scientific, BP232-1), 17 UM Pantothenate (Fisher Scientific, AAA1660922), 1 μM Insulin (sigma, 19278), (Fisher Scientific, AAA1991006), and 1 μM Dexamethasone (Fisher Scientific, D16911G) then sterile filtered through a 0.22 μM PES filter bottle. Cells are then exposed to freshly prepared Adipocyte Maintenance Media and cultured for 4 days.

After 7 days of differentiation, cells are characterized for adipogenic differentiation. Adipogenic differentiation is assessed by the presence of intracellular lipid droplets by Oil Red O staining. Specifically, the cells are fixed in 10% (v/v) neutral buffered formaldehyde (Sigma, HT501128) for 1 h and stained for 10 min with a 60% (v/v) Oil Red O solution (Fisher, AAA1298914). The rate of differentiation is expressed as the ratio of the number of Oil Red O-positive cells to the number of total cells.

The efficiency of adipogenic differentiation is also quantified via flow cytometric analysis. Specifically, LipidTOX Deep Red (Fisher, H34477) is added to the cell suspension at 1:200 dilution and mixed gently. The cells are incubated at room temperature for 30 min. The cells are then analyzed on a flow cytometer. It is expected that differentiated adipocytes are stained for LipidTOX at a higher level compared to ASCs.

The levels of adipocyte-specific gene expressions in the differentiated cells are quantified by qPCR. Total RNA is isolated, cDNA generated, and qRT-PCR performed for adipogenic genes including Adiponectin, Pparg, Leptin, Lipoprotein lipase, FABP4. GAPDH and Actin are used as controls.

Overall, this example demonstrates, inter alia, the ability to manufacture the dual-layer hydrogel polymer enclosure containing ASCs for the purpose of expansion, differentiation to adipocytes, and characterization.

### Example 6: In Vitro Testing of Dual-Layer Hydrogel Encapsulation of Adipocytes

This example demonstrates, inter alia, the in vitro activity of encapsulated ASCs after differentiation into adipocytes by measuring secretion of leptin, secretion of lipoprotein lipase, and BCAA catabolism.

The encapsulated cells generated in Example 5 are evaluated for in vitro leptin secretion by leptin ELISA (BioLegend, Cat #: 444304), and lipoprotein lipase (LPL) secretion by LPL ELISA (ABclonal, Catalog number: RK06714). Determination of branched-chain alpha-keto acid dehydrogenase activity is performed as previously described (Nakai et al., 2000; PMID: 10989417) on cell lysate of cells generated in Example 5, as well as for HepG2 cells as a positive control. In brief, activity is measured using an assay that monitors NADH production over time. Activity is then normalized to total protein added to the reaction using a BCA assay (ThermoFisher, BCA Protein Assay Kit). In addition, branched chain amino acid (BCAA) depletion is measured over time from cells generated in Example 5, as well as HepG2 cells, by incubating cells with cell culture media (DMEM) and taking samples of cell culture media at 4 hours and 24 hours. DMEM without cells serve as a negative control. BCAA concentration is then measured using an enzymatic assay (Sigma, Catalog number: MAK003).

It is expected that adipocytes differentiated in dual-layer hydrogel encapsulation will secrete appreciable levels of leptin and lipoprotein lipase when assayed in vitro, as well as show appreciable levels of BCAA catabolism.

### Example 7: In Vitro Testing of Dual-Layer Hydrogel Encapsulation of ASCs

This example demonstrates, inter alia, the in vitro activity of encapsulated ASCs by measuring BCAA catabolism.

The encapsulated cells generated in Example 4 are evaluated for BCAA catabolismactivity. Determination of branched-chain alpha-keto acid dehydrogenase activity is performed as previously described (Nakai et al., 2000; PMID: 10989417) on cell lysate of cells generated in Example 4, as well as for HepG2 cells as a positive control. In brief, activity is measured using an assay that monitors NADH production over time. Activity is then normalized to total protein added to the reaction using a BCA assay (ThermoFisher, BCA Protein Assay Kit). In addition, branched chain amino acid (BCAA) depletion is measured over time from cells generated in Example 4, as well as HepG2 cells, by incubating cells with cell culture media (DMEM) and taking samples of cell culture media at 4 hours and 24 hours. DMEM without cells serve as a negative control. BCAA concentration is then measured using an enzymatic assay (Sigma, Catalog number: MAK003).

Without being bound to a particular theory, it is expected that ASCs in dual-layer hydrogel encapsulation will show appreciable levels of BCAA catabolism.

### Example 8: Therapeutic Effects of Dual-Layer Hydrogel Encapsulated Adipocytes in a Mouse Model of Lipodystrophy and Leptin-Deficiency

This example demonstrates, inter alia, in vivo activity of the encapsulated cells after differentiation into adipocytes.

In this example, the encapsulated cells generated in Example 5 are evaluated for their in vivo activity in a mouse model of lipodystrophy and leptin-deficiency (the ob/ob mouse, Jax strain #: 000632). The adipocytes differentiated in Example 5 are implanted subcutaneously into the ob/ob mice, with a cell dose of 20-140 million per mouse. As a control, another group of ob/ob mice are injected in the same manner with HBSS only, without cells. Body weight, food intake, plasma triglycerides (ThermoFisher, Catalog number: TR22421), plasma insulin (Crystal Chem, Catalog number: 900080), and non-fasting glucose (glucometer) is then measured over time following administration of the cells or HBSS. In addition, a glucose tolerance test (GTT) is also performed at Day 38 after administration of cells or HBSS. In brief, the GTT involves an intraperitoneal injection of dextrose (1 mg/kg of body weight) after fasting mice for 6 hours and then measuring blood glucose levels from the tail at 0, 15-, 30-, 60-, 90-, and 120-minutes post glucose injection using a glucometer. GTT data is analyzed by area under the curve (AUC) as is well-described in the literature for a GTT (Virtue et al., 2021: PMID: 34117483).

### Example 9: Therapeutic Effects of Dual-Layer Hydrogel Encapsulated Adipocytes or ASCs in a Mouse Model of Maple Syrup Urine Disease (MSUD

This example demonstrates, inter alia, in vivo activity of the encapsulated ASCs or adipocytes.

In this example, the encapsulated cells generated in Example 4 or Example 5 are evaluated for their in vivo activity in a mouse model of maple syrup urine disease (MSUD) (e.g. Jax strain #: 006999). Either the ASCs generated from Example 4, or the adipocytes differentiated in Example 5 are implanted subcutaneously into the MSUD mice, with a cell dose of 20-140 million per mouse. As a control, another group of MSUD mice are injected in the same manner with HBSS only, without cells. Plasma BCAAs are the then measured (Sigma, Catalog number: MAK003), and survival followed over time.

Without being bound to a particular theory, it is expected that the transplanted cells will reduce plasma BCAA levels and extend survival.

### REFERENCES

Gramignoli, R.; Green, M. L.; Tahan, V.; Dorko, K.; Skvorak, K. J.; Marongiu, F.; Zao, W.; Venkataramanan, R.; Ellis, E. C.; Geller, D.; Breite, A. G.; Dwulet, F. E.; McCarthy, R. C.; Strom, S. C. Development and application of purified tissue dissociation enzyme mixtures for human hepatocyte isolation. Cell Transplant. 21 (6): 1245-1260; 2012.
Gramignoli, R.; Dorko, K.; Tahan, V.; Skvorak, K. J.; Ellis, E.; Jorns, C.; Ericzon, B. G.; Fox, I. J.; Strom, S. C. Hypothermic storage of human hepatocytes for transplantation. Cell Transplant. 23 (9): 1143-1151; 2014.
Mossanen, J. C.; Tacke, F.; Acetaminophen-induced acute liver injury in mice. Laboratory Animals 49 (1): 30-36; 2015.
Viswanathan, P.; Sharma, Y.; Jaber, F.-L.; Tchaikovskaya, T.; Gupta, S. Transplanted hepatocytes rescue mice in acetaminophen-induced acute liver failure through paracrine signals of hepatic ATM and STAT3 pathways. FASEB J. 35 (4): e21471; 2021.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

As used herein, all headings are simply for organization and are not intended to limit the disclosure in any manner. The content of any individual section may be equally applicable to all sections.

### INCORPORATION BY REFERENCE

All patents and publications referenced herein are hereby incorporated by reference in their entireties.

What is claimed is:

1. A dual-layer, semi-permeable, fibrosis-resistant, hydrogel polymer enclosure comprising:
a. an exterior hydrogel layer encompassing the enclosure, optionally comprising one or more rejection inhibition agents that reduce rejection upon implantation in a mammalian subject;
b. an interior hydrogel layer disposed within the exterior hydrogel layer, optionally comprising one or more agents that promote cell viability, cell differentiation, cell health, cell function, and/or reduction and/or prevention and/or inhibition of apoptosis, and/or preventing rejection and/or fibrosis, further optionally wherein the exterior hydrogel layer and the interior hydrogel layer are separated by a liquid layer, such as a culture medium; and
c. an interior aqueous chamber disposed in the interior hydrogel layer, wherein the interior aqueous chamber is suitable for housing at least about 10 living mammalian cells, optionally where the volume allows the at least about 10 cells to proliferate within the chamber,
wherein the hydrogel polymer enclosure is suitable for administration to a human subject.

2. The hydrogel polymer enclosure of claim 1, wherein the interior and exterior hydrogel layers independently comprise an alginate, a methacrylate, or a combination thereof.

3. The hydrogel polymer enclosure of claim 1, wherein the interior and/or exterior hydrogel layers comprise an alginate or methacrylate, optionally wherein the alginate or methacrylate comprises one or more zwitterionic groups such as phosphorbetaine, sulfobetaine, carboxybetaine, cysteine, sulfopyridinium betaine, phosphorylcholine, or sulfobetain siloxane.

4. The hydrogel polymer enclosure of any one of the preceding claims, comprising cells in the interior aqueous chamber, optionally wherein the cells are mammalian cell, such as human cells, including cells derived from human induced pluripotent stem cells (iPSCs), adipose-derived stem cells (ASCs), or embryonic stem cells (ESCs).

5. The hydrogel polymer enclosure of claim 4, wherein the cells within the interior aqueous chamber are iPSCs, ASCs, or ESCs and maintain an ability to divide without significant loss or reduction of genetic stability (e.g., minimization of: copy number variation (CNV) or emergence of single-nucleotide polymorphisms (SNPs)) while maintained in culture, optionally as evaluated by assays such as karyotyping, restriction endonuclease mapping, ddPCR, and/or DNA sequencing.

6. The hydrogel polymer enclosure of claim 4, wherein the cells within the interior aqueous chamber are iPSCs, ASCs, or ESCs and are capable of stably differentiating into mesoderm, endoderm, or ectoderm lineages.

7. The hydrogel polymer enclosure of any one of the preceding claims, wherein the interior hydrogel layer comprises one or more agents that promote cell viability and/or function, optionally wherein the one or more agents that promote cell viability and/or function are polypeptides that have a biological function promoting genetic stability and/or facilitating differentiation, including one or more of collagen V A1, collagen V A2, collagen VI A1, collagen VI A2, collagen VI A3, fibronectin, a laminin, fibrin, fibrinogen alpha, fibrinogen beta, fibrinogen gamma, factor XIII a chain, factor XIII b chain, basement membrane-specific heparan sulfate proteoglycan core protein, and elastin.

8. The hydrogel polymer enclosure of any one of the preceding claims, wherein the exterior hydrogel layer comprises one or more rejection inhibition agents, optionally wherein the one or more rejection inhibition agents are zwitterionic groups, optionally wherein the zwitterionic groups prevent undesirable protein adsorption, e.g., via formation of a hydration shell.

9. The hydrogel polymer enclosure of claim 8, wherein the zwitterionic groups are selected from sulfobetaine, carboxybetaine, phosphocoline, or other anti-fouling polymers, including combinations of the foregoing.

10. The hydrogel polymer enclosure of claim 8, wherein the exterior hydrogel comprises polyethylene glycol (PEG) linkers.

11. The hydrogel polymer enclosure of claim 8, wherein the exterior hydrogel layer reduces, eliminates, or mitigates undesirable foreign body reactions including macrophage attachment and lymphocyte activation.

12. The hydrogel polymer enclosure of any one of the preceding claims comprising an extracellular matrix (ECM) component, such as one or more of: reconstituted basement membrane (e.g., Matrigel), collagen, fibronectin, laminin, fibrin, nestin, perlecan, or signaling domains such as RGD, including combinations thereof.

13. The hydrogel polymer enclosure of any one of the preceding claims, wherein the enclosure is substantially spherical.

14. The hydrogel polymer enclosure of claim 13, wherein the substantially spherical enclosure has a diameter of about: 200-1000 μm, e.g., 300-800 μm, 400-600 μm, 450-550 μm.

15. The hydrogel polymer enclosure of any one of the preceding claims, wherein the interior aqueous chamber has a volume suitable for at least about: 10, 20, 30, 40, 50, 60, 70, 80, 90, 100, 150, 200, 250, 300, 350, 400, 450, 500, 550, 600, 650, 700, 750, 800, 850, 900, 950, 1000, 1250, 1500, 2000, 2500, 3000, 3500, 4000, 4500, 5000, 6000, 7000, 8000, 9000, 10000, 11000, 12000, 13000, 14000, 15000, 16000, 17000, 18000, 19000, 20000, or more mammalian cells (such as iPSCs, ASCs, ESCs, or cells differentiated from any of the foregoing, such as hepatocytes).

16. The hydrogel polymer enclosure of any one of the preceding claims, wherein the interior aqueous chamber comprises at least about: 10, 20, 30, 40, 50, 60, 70, 80, 90, 100, 150, 200, 250, 300, 350, 400, 450, 500, 550, 600, 650, 700, 750, 800, 850, 900, 950, 1000, 1250, 1500, 2000, 2500, 3000, 3500, 4000, 4500, 5000, 6000, 7000, 8000, 9000, 10000, 11000, 12000, 13000, 14000, 15000, 16000, 17000, 18000, 19000, 20000, or more mammalian cells (such as iPSCs, ASCs, ESCs, or cells differentiated from any of the foregoing, such as hepatocytes), optionally wherein the mammalian cells are genetically transformed (transiently or stably), further wherein the genetic transformation is expression of a transgene (e.g., a therapeutic protein, such as one or more of: an enzyme (including a gene editing system), a growth factor, a ligand, an antibody, or a structural protein; or a therapeutic nucleic acid, such as an siRNA or an aptamer).

17. The hydrogel polymer enclosure of claim 16, wherein cells maintain at least about: 80, 85, 90, 91, 92, 93, 94, 95, 96, 97, 98, or 99% viability while maintaining no copy number variation over at least about:
10, 12, 15, 20, 25, 30, or more, doublings in culture (e.g., from about 1 L to 2000 L, or more) as measured via karyotyping, ddPCR, sequencing, or other assays.

18. The hydrogen polymer enclosure of claim 16 or 17, wherein at least about: 80, 85, 90, 91, 92, 93, 94, 95, 96, 97, 98, or 99%, or more, of the cells are negative for a cell death signal, such as an apoptotic or pre-apoptotic signal (such as activated Caspase 3 or Annexin V staining), e.g., over at least about: 10, 12, 15, 20, 25, 30, or more, doublings in culture (e.g., from about 1 L to 2000 L, or more) or upon administration to a subject, over at least about: 5, 10, 20, 30 days, or more, e.g., 5, 10, 15, 20 weeks.

19. The hydrogel polymer enclosure of claims 16-18 wherein cells maintain at least about: 80, 85, 90, 91, 92, 93, 94, 95, 96, 97, 98, or 99% genetic stability as measured via karyotyping ddPCR, sequencing, or other assays during expansion over at least about: 10, 12, 15, 20, 25, 30, or more, doublings in culture (e.g., from about 1 L to 2000 L, or more).

20. The hydrogel polymer enclosure of any one of claims 16-19, wherein, upon implantation in a mammalian subject, the enclosure elicits, relative to controls (unenclosed cells or enclosures that are not fibrosis-resistant), reduced levels of one or more of: macrophage attachment, cell-derived innate immune system response, or lymphocyte activation.

21. The hydrogel polymer enclosure of any one of claims 16-20, wherein, upon implantation in a mammalian subject, the enclosure does not elicit an immune response.

22. The hydrogel polymer enclosure of any one of the preceding claims, wherein the exterior hydrogel layer has an average thickness of about: 5-50 μm, e.g., about: 10-50 μm, such as about: 10-20 μm, e.g., about: 5, 10, 15, 20, 5, 30, 35, 40, 45, 50, 55, or 60 μm.

23. The hydrogel polymer enclosure of any one of the preceding claims, wherein the interior hydrogel layer has an average thickness of about: 5-50 μm, e.g., about: 10-50 μm, such as about: 10-20 μm, e.g., about: 5, 10, 15, 20, 5, 30, 35, 40, 45, 50, 55, or 60 μm.

24. The hydrogel polymer enclosure of any one of the preceding claims, wherein the interior hydrogel layer and/or exterior hydrogel layer has an average pore size of about: 1-20 μm, e.g., about: 0.1, 0.25, 0.5, 0.75, 1, 2, 3, 4, 5, 6, 7, 8, 9, 10, 12, 13, 14, 15, 16, 17, 18, 19, or 20 μm, or about 1-10 μm, such as about 5 μm.

25. The hydrogel polymer enclosure of any one of the preceding claims, wherein the interior hydrogel layer and/or exterior hydrogel layer has an average pore size to allow molecules less than about: 50, 60, 70, 80, 90, 100, 150, 200, 250, 300, 350, 400, 500, 600, 700, 800, 900, or 1000 kDa to pass, but not molecules larger than about 1000 kDa.

26. The hydrogel polymer enclosure of any one of the preceding claims, wherein the enclosure is made by a method comprising coaxial jetting comprising an outer steam comprising a polymer solution and an interior steam comprising cells, optionally wherein the method is performed under cGMP conditions (21 CFR Parts 210, 211, 314) and/or International Council on Harmonization (ICH) quality guidelines (Q7 and others).

27. The hydrogel polymer enclosure of any one of the preceding claims, wherein the cell differentiation comprises differentiation of a stem cell to a primary cell.

28. A composition comprising a plurality of the hydrogel polymer enclosures of any one of the preceding claims.

29. The composition of claim 28, wherein the hydrogel polymer enclosures have a coefficient of variation of less than about: 15, 14, 13, 12, 11, 10, 9, 8, 7, 6, 5, 4, 3, 2, or 1%.

30. The composition of claim 28 or 29, wherein at least about: 60, 70, 80, 85, 90, 95%, or more of the hydrogel polymer enclosures contain at least about: 1, 2, 3, 4, 5, 10, 15, 20, 25, 30, 40, 50, 100, 200, 250, 500, 550, 600, 650, 700, 750, 800, 850, 900, 950, 1000, 1250, 1500, 2000, 2500, 3000, 3500, 4000, 4500, 5000, 6000, 7000, 8000, 9000, or more, mammalian cells.

31. The composition of any one of claims 28-30, wherein the composition is sterile (free of fungal, bacterial, or archaeal cells; free of viral particles), pyrogen-free, substantially free of debris, or a combination of any or all of the foregoing.

32. A method for delivering one or more cells, the method comprising contacting a biological tissue with a hydrogel polymer enclosure or composition of any one of the preceding claims, wherein the hydrogel polymer enclosure or composition comprises one or more cells, such as mammalian cells.

33. The method of claim 32, wherein the biological tissue is in a mammalian subject.

34. A method for treating a disease or disorder, the method comprising providing a therapeutically effective amount of a hydrogel polymer enclosure or composition of any one of the preceding claims to a tissue in a subject in need thereof, wherein the hydrogel polymer enclosure or composition comprises one or more cells, such as mammalian cells.

35. The method of claim 34, wherein the tissue is a liver tissue and the one or more cells are hepatocytes (including organoid-associated hepatocytes), such as iPSC-derived, ASC-derived, or ESC-derived hepatocytes.

36. The method of claim 34 or 35, wherein the mammalian subject has, or is suspected of having, a liver disease, such as acute liver failure (ALF; including those due to or related to inherited metabolic diseases [IMDs]), chronic liver failure, and acute-on-chronic liver failure (ACLF).

37. The method of claim 34 or 35, wherein the mammalian subject has, or is suspected of having, a disease resulting in deficient liver function or functions selected from:
a) a disease resulting in deficient synthesis of a protein (e.g., alpha 1 antitrypsin deficiency, Wilson's disease) or multiple proteins by the liver,
b) a disease resulting in deficient metabolic function or functions of the liver (e.g., ornithine transcarbamylase deficiency, Crigler-Najjar syndrome), or
c) a disease resulting in deficiencies in multiple functions of the liver (e.g., the various forms of liver failure, acute, chronic, and acute on chronic).

38. A method of culturing cells, comprising culturing, in a bioreactor, a hydrogel polymer enclosure or composition of any one of the preceding claims, wherein the hydrogel polymer enclosure or composition comprises one or more cells.

39. The composition of claim 30 or 31 or the method of any one of claims 32-38, wherein the cells:
a. are dispersed on a microcarrier;
b. were previously cultured in the interior of a semi-permeable enclosure (such as an alginate enclosure), before disassociating the semi-permeable enclosure, recovering the cells, and re-enclosing the cells in the hydrogel polymer enclosure of any of the preceding claims;
c. were recovered from cryopreservation; or
d. a combination of the foregoing.

40. The composition of claim 30 or 31 or the method of any one of claims 32-39, wherein the hydrogel polymer enclosure was recovered from cryopreservation.

41. The hydrogel polymer enclosure of any one of claims 1-27, compositions of any one of claim 28-31, 39, or 40, wherein the composition is suitable for cryopreservation, wherein the composition optionally further comprises one or more of: DMSO, glycerol, an anti-apoptosis compound (e.g., ZVAD), an iron chelator (e.g., desferoxamine), serum albumin (such as human serum albumin), or a combination of the foregoing.

42. A method comprising cooling the enclosure or composition of claim 41 to about: −20, −30, −40, −50, −60, −70, −80, −90, −100, −110, −120, −130, −140, −150, −160, −170, −180, −190 degrees Celsius.

43. A dual-layer, semi-permeable, fibrosis-resistant, hydrogel polymer enclosure comprising:
a. an exterior hydrogel layer encompassing the enclosure, optionally comprising one or more rejection inhibition agents that reduce rejection upon implantation in a mammalian subject;
b. an interior hydrogel layer disposed within the exterior hydrogel layer, the interior hydrogel layer comprising alginate and comprising one or more agents that promote cell viability, cell differentiation (e.g., of a stem cell to a primary cell), cell health, cell function, and/or reduction and/or prevention and/or inhibition of apoptosis, and/or preventing rejection and/or fibrosis, further optionally wherein the exterior hydrogel layer and the interior hydrogel layer are separated by a liquid layer, such as a culture medium; and
c. an interior aqueous chamber disposed in the interior hydrogel layer, wherein the interior aqueous chamber comprises at least about 10 living induced pluripotent stem cells (iPSCs),
wherein the hydrogel polymer enclosure is suitable for administration to a human subject.

44. A dual-layer, semi-permeable, fibrosis-resistant, hydrogel polymer enclosure comprising:
a. an exterior hydrogel layer encompassing the enclosure, optionally comprising one or more rejection inhibition agents that reduce rejection upon implantation in a mammalian subject;
b. an interior hydrogel layer disposed within the exterior hydrogel layer, the interior hydrogel layer comprising alginate and comprising one or more agents that promote cell viability, cell differentiation (e.g., of a stem cell to a primary cell), cell health, cell function, and/or reduction and/or prevention and/or inhibition of apoptosis, and/or preventing rejection and/or fibrosis, further optionally wherein the exterior hydrogel layer and the interior hydrogel layer are separated by a liquid layer, such as a culture medium; and
c. an interior aqueous chamber disposed in the interior hydrogel layer, wherein the interior aqueous chamber comprises at least about 10 living embryonic stem cells (ESCs),
wherein the hydrogel polymer enclosure is suitable for administration to a human subject.

45. A dual-layer, semi-permeable, fibrosis-resistant, hydrogel polymer enclosure comprising:
a. an exterior hydrogel layer encompassing the enclosure, optionally comprising one or more rejection inhibition agents that reduce rejection upon implantation in a mammalian subject;
b. an interior hydrogel layer disposed within the exterior hydrogel layer, the interior hydrogel layer comprising alginate and comprising one or more agents that promote cell viability, cell differentiation (e.g., of a stem cell to a primary cell), cell health, cell function, and/or reduction and/or prevention and/or inhibition of apoptosis, and/or preventing rejection and/or fibrosis, further optionally wherein the exterior hydrogel layer and the interior hydrogel layer are separated by a liquid layer, such as a culture medium; and
c. an interior aqueous chamber disposed in the interior hydrogel layer, wherein the interior aqueous chamber comprises at least about 10 living adipose derived stem cells (ASCs),
wherein the hydrogel polymer enclosure is suitable for administration to a human subject.

46. The hydrogel polymer enclosure of any one of claims 43-45, wherein the volume allows at least about 10 cells to proliferate within the chamber.

47. The hydrogel polymer enclosure of any one of claims 43-45, wherein the one or more agent is or comprises extracellular matrix (ECM) proteins.

48. The hydrogel polymer enclosure of any one of claims 43-45, wherein the cells are capable of stably differentiating into mesoderm, endoderm, or ectoderm lineages.

49. A dosage form of a therapeutically effective amount of the hydrogel polymer enclosure of any one of claims 1-27, or composition of any one of claim 28-31, 39 or 40.

50. The dosage form of claim 49, wherein the dosage form is a container, optionally a syringe.

51. The dosage form of claim 49 or 50, wherein the hydrogel polymer enclosure or composition further comprises a pharmaceutically acceptable carrier, diluent, excipient, or vehicle.

52. The hydrogel polymer enclosure of any one of claim 1-27, 41, or 43-48, the composition of any one of claim 28-31, 39, or 40, or method of any one of claims 32-38, 42, wherein the enclosure was made by microfluidic co-axial jetting.
